(19) United States
(12) Patent Application Publication
(10) Pub. No.: US 2020/0342955 A1
(43) Pub. Date: Oct. 29, 2020

(54) PREDICTING CANCER-RELATED PATHOGENIC IMPACT OF SOMATIC MUTATIONS USING DEEP LEARNING-BASED METHODS

(71) Applicant: APOSTLE, INC., Sunnyvale, CA (US)

(72) Inventors: Xin GUO, Palo Alto, CA (US); Dongliang GE, Redwood City, CA (US); Bo ZHANG, Palo Alto, CA (US)

(21) Appl. No.: 16/758,374

(22) PCT Filed: Oct. 29, 2018
(86) PCT No.: PCT/US2018/058038

§ 371 Date: Apr. 22, 2020

Related U.S. Application Data
(60) Provisional application No. 62/578,330, filed on Oct. 27, 2017.

(51) Int. Cl. G16B 20/20; G16B 50/10; G16B 40/00; C12Q 1/6886; G16H 70/60; G16H 50/20; G16H 50/30

(52) U.S. Cl. CPC G16B 20/20; G16B 50/10; G16B 40/00; C12Q 2600/156; G16H 70/60; G16H 50/20; G16H 50/30; C12Q 1/6886

(57) ABSTRACT

Cancer is a genetic disease initiated by somatic mutations and progressed by an accumulation of genomic aberrations. Differentiating cancer driver somatic mutations from passenger and benign mutations is a critical step toward better understanding of cancer biology. It also provides important insights into cancer detection and prognosis monitoring. Provided herein are machine learning methods that utilize a deep-learning framework to predict mutation-associated pathogenicity, including cancer-related pathogenicity risk of somatic mutations. The methods incorporate not only an annotation comprising functional features, genomic features, epigenetic features, and other annotated features related to the mutation, but also a separate annotation including the surrounding sequence content of the test mutation. The methods can provide a quantitative score from the two or more annotation sets of a mutant reflecting the pathogenic risk of a mutation, including those involved in carcinogenesis and cancer progression.

### CROSS-REFERENCE TO RELATED APPLICATIONS

This application claims the benefit of U.S. Provisional Application No. 62/578,330, filed Oct. 27, 2017, the content of which is incorporated herein by reference in its entirety.

### FIELD OF ART

The disclosure relates to deep-learning based machine learning methods to predict pathogenicity of a given genomic sequence variation.

### BACKGROUND

Cancer is a genetic disease characterized by a progressive accumulation of genomic aberrations. When somatic mutations arise in single cells at certain key genetic regions, those cells may lose necessary controls during the cell replication cycles, eventually leading to uncontrolled proliferation and tumorigenesis.

All cells, including cancer cells, contain somatic mutations, but not all somatic mutations can cause cancer. Throughout a person's life, somatic mutations occur spontaneously in somatic cells, and the majority of these somatic mutations do not have a noticeable functional effect. Only a small fraction of somatic mutations are capable of altering key cellular functions that contribute to neoplasia. These cancer-related mutations may be a direct cause of cancer, or facilitate cancer development, or may cause or facilitate cancer metastasis. Such mutations are termed “cancer-related mutations” or “cancer driver mutations”. By contrast, the term “passenger or benign somatic mutations” refers to mutations that have little causal effect on the fitness of a cellular clone.

Previous studies have demonstrated some common genetic and functional characteristics of cancer-related somatic mutations. Cancer driver mutations often reside in protein-coding regions or gene regulatory regions and affect protein folding and stability or gene regulatory control and subcellular localization. For example, driver mutations in protein-coding regions of oncogenes tend to be missense mutations at specific codons or focal amplifications. Nonsense or frameshift mutations or focal deletions are often the hallmark of cancer-driver mutations in tumor suppressors. Passenger or benign mutations, on the other hand, are believed to have neutral functional impact on cell phenotype.

Cancer driver mutations are often identified by studying genomic profiles of a cohort of tumor samples using high throughput sequencing technology, based on the assumption that cancer-causing mutations occur more frequently in tumor samples than the background variation frequency. However, patterns of cancer driver mutations can differ widely by tissue of origin or from patient to patient and even in different sites of the same tumors. Furthermore, recurrence may not necessarily mean relatedness to cancer. Mutations with high frequency may indicate that they are related to molecular pathways associated with tumor, or they could be the by-product genetic alterations during the tumor progression. Similarly, low frequency of passenger mutations may indicate potential deleterious effects on cancer cells as well. For instance, accumulation of moderately deleterious passenger mutations can alter the course of cancer progression and lead to several oncological phenomena.

Distinguishing cancer-related somatic mutations from passenger or benign mutations improves the ability to effectively and accurately diagnose cancer and monitor cancer progression. What is needed, therefore, are improved methods to predict cancer-related pathogenicity risk of, or other types of pathogenicity corresponding to, specific somatic mutations.

### SUMMARY

Embodiments of the invention comprise machine learning methods that utilize a deep-learning framework to predict cancer-related pathogenicity risk of somatic mutations. The methods incorporate not only an annotation of associated functional, genomic, and epigenomic and other related features, but also an annotation of the surrounding sequence content (e.g., DNA flanking sequence) of the test mutation. The output of the methods is a quantitative score evaluating the pathogenic risk of the mutation in carcinogenesis and cancer progression.

According to some embodiments, provided herein is a computer-implemented method for predicting a pathogenicity of a test genomic mutation, the method comprising i) preparing input data for said test genomic mutation, comprising generating a first representation comprising a primary annotation of said test genomic mutation, wherein said primary annotation comprises a nucleotide sequence surrounding said test genomic mutation; and generating a second representation comprising at least one feature of a secondary annotation of said test genomic mutation, wherein said feature comprises a functional feature, a genomic feature, or an epigenomic feature; and ii) generating a score from said first and second representations of said test genomic mutation using a convolutional neural network (CNN), wherein said convolutional neural network comprises a first sub-CNN network configured to process the first representation, a second sub-CNN network configured to process the second representation, and a third sub-CNN network configured to process the output of the first and second sub-CNN network, wherein generating said score comprises: receiving, by the first sub-CNN network, said first representation of said test genomic mutation; generating, by the first sub-CNN network, output comprising a first feature vector; receiving, by the second sub-CNN network, said second representation of said test genomic mutation; generating, by the second sub-CNN network, output comprising a second feature vector; receiving, by the third sub-CNN network, input comprising the first feature vector and the second feature vector; and generating, by the third sub-CNN network, output representing a score indicative of a pathogenicity associated with the test genomic mutation.

In some embodiments, the third sub-CNN network comprises a fully connected layer. In some embodiments, the pathogenicity is a cancer risk from said mutation.

In some embodiments, the method further comprises generating a third representation comprising at least one feature of said secondary annotation of said test genomic mutation. In some embodiments, the method further comprises receiving, by the third sub-CNN network, input comprising the third representation. In some embodiments, the third representation is a vector. In some embodiments, the vector comprises functional features corresponding to said mutation.

In some embodiments, the second representation is a matrix. In some embodiments, the matrix comprises genomic or epigenomic feature information corresponding to said surrounding nucleotide sequence. In some embodiments, the first representation is a vector. In some embodiments, the vector comprises said surrounding nucleotide sequence.

In some embodiments, the convolutional neural network is trained based on training data comprising: a first data set comprising pathogenic mutations, and a second data set comprising benign mutations. In some embodiments, the pathogenic mutations are cancer driver mutations. In some embodiments, the training data comprises said first representation and said second representation for each mutation in said first data set and said second data set. In some embodiments, the training data further comprises said third representation for each mutation in said first data set and said second data set.

In some embodiments, the method further comprises storing said test genomic mutation in a database if said score indicates a high pathogenicity risk associated with the mutation.

In some embodiments, the method further comprises training a convolutional neural network based on a training data set, said training data comprising: a first data set comprising said first representation and said second representation for each of a plurality of pathogenic mutations, and a second data set comprising said first representation and said second representation for each of a plurality of benign mutations. In some embodiments, the first data set and said second data set each further comprise said third representation.

Also provided herein, according to some embodiments, is a computer-implemented method for predicting a pathogenicity of a test genomic mutation, the method comprising: i) preparing input data for said test genomic mutation, comprising: generating a first representation comprising a primary annotation of said test genomic mutation, wherein said primary annotation comprises a nucleotide sequence surrounding said test genomic mutation; generating a second representation and a third representation each comprising one or more features of a secondary annotation of said test genomic mutation, wherein said secondary annotation comprises functional, genomic, or epigenomic features, wherein said second representation comprises features corresponding to said nucleotide sequence surrounding said test genomic mutation, wherein said third representation comprises functional features corresponding to said test genomic mutation; ii) generating a score from said first, second, and third representations of said test genomic mutation using a convolutional neural network (CNN), wherein said convolutional neural network comprises a first sub-CNN network configured to process the first representation, a second sub-CNN network configured to process the second representation, and a third sub-CNN network configured to process the output of the first and second sub-CNN network and the third representation, wherein generating said score comprises: receiving, by the first sub-CNN network, said first representation of said test genomic mutation; generating, by the first sub-CNN network, output comprising a first feature vector; receiving, by the second sub-CNN network, said second representation of said test genomic mutation; generating, by the second sub-CNN network, output comprising a second feature vector; receiving, by the third sub-CNN network, input comprising the first feature vector, the second feature vector, and said third representation of said test genomic mutation; and generating, by the third sub-CNN network, output representing a score indicative of a pathogenicity associated with the test genomic mutation.

Also provided herein, according to some embodiments, is a computer-implemented method of predicting a pathogenicity score of a test genomic mutation, comprising: preparing input data for said test genomic mutation, comprising generating a first representation comprising a primary annotation of said test genomic mutation, wherein said primary annotation comprises a nucleotide sequence surrounding said test genomic mutation; and generating a second representation comprising a feature of a secondary annotation of said test genomic mutation, wherein said secondary annotation comprises functional, genomic, or epigenomic features; and providing the first and second representation as input to a trained machine learning model, wherein the machine learning model is configured to receive input comprising said first representation and said second representation and generate a score indicative of a pathogenicity risk associated with the test genomic mutation. In some embodiments, the score is a cancer risk score.

In some embodiments, preparing said input data further comprises generating a third representation comprising a functional feature of said secondary annotation. In some embodiments, the method further comprises providing the third representation as input to said trained machine learning model, said machine learning model configured to receive said third representation as input.

In some embodiments, the first representation is a vector comprising said surrounding nucleotide sequence. In some embodiments, the second representation is a vector. In some embodiments, the second representation is a matrix. In some embodiments, the matrix comprises epigenomic or genomic feature information corresponding to said surrounding nucleotide sequence. In some embodiments, the third representation is a vector. In some embodiments, the vector comprises functional features corresponding to said mutation.

In some embodiments, the machine learning model is trained based on training data comprising: a first data set comprising pathogenic mutations; and a second data set comprising benign mutations. In some embodiments, the pathogenic mutations are cancer driver mutations.

In some embodiments, the training data comprises said first representation and said second representation for each mutation in said first data set and said second data set. In some embodiments, the training data set comprises said third representation for each mutation in said first data set and said second data set.

In some embodiments, the method further comprises storing said test genomic mutation in a database if said score indicates a high cancer risk associated with the mutation.

In some embodiments, the method further comprises training a machine learning model based on a training data set, said training data comprising: a first data set comprising said first representation and said second representation for each of a plurality of pathogenic mutations, and a second data set comprising said first representation and said second representation for each of a plurality of benign mutations. In some embodiments, the pathogenic mutations are cancer driver mutations. In some embodiments, the first and second data set further comprise said third representation for each of said mutations.

Also provided herein, according to some embodiments, is a computer-implemented method of predicting a cancer risk score of a test genomic mutation, comprising: i) preparing input data for said test genomic mutation, comprising: generating a first representation comprising a primary annotation of said test genomic mutation, wherein said primary annotation comprises a nucleotide sequence surrounding said test genomic mutation; generating a second representation comprising one or more features of a secondary annotation of said test genomic mutation, wherein said secondary annotation comprises functional, genomic, or epigenomic features corresponding to said nucleotide sequence surrounding said test genomic mutation; generating a third representation comprising one or more functional features of said secondary annotation of said test genomic mutation; and ii) providing the first, second, and third representations as input to a trained machine learning model, wherein the machine learning model is configured to: receive input comprising said first representation and said second representation, and generate a score indicative of a cancer risk associated with the test genomic mutation.

In some embodiments, the methods described herein use said score to identify said cancer risk associated with the test genomic mutation. In some embodiments, the score is used to identify a cancer driver mutation.

In some embodiments, the methods described herein further comprise repeating said method to identify a plurality of cancer driver mutations. In some embodiments, the methods described herein further comprise sending said score for display via a user interface. In some embodiments, the methods described herein further comprise transforming the score to a defined range using a sigmoid function. In some embodiments, the defined range is from 0 to 1.

In some embodiments, the pathogenicity is a cancer risk associated with a specific tissue type. In some embodiments, the pathogenicity is a cancer risk associated with a specific cancer type. In some embodiments, the pathogenicity is a cancer risk that is predictive of the probability that a mutation is causative for cancer.

In some embodiments, the methods described herein further comprise performing said method on a plurality of test genomic mutations.

In some embodiments, the test genomic mutation is a human genomic mutation. In some embodiments, the test genomic mutation is a somatic mutation. In some embodiments, the test genomic mutation comprises a missense genomic mutation, a nonsense genomic mutation, a splice-site genomic mutation, an insertion genomic mutation, a deletion genomic mutation, or a regulatory element genomic mutation. In some embodiments, the test genomic mutation comprises a single nucleotide polymorphism.

In some embodiments, the secondary annotation comprises a protein level functional impact feature, a region identifier feature, a sequence conservation feature, an epigenetic status feature, a genomic feature of the surrounding sequence, or any combination thereof.

Also provided herein, according to some embodiments, is a non-transitory computer-readable storage medium comprising computer-executable instructions for carrying out any one of the above claims.

Also provided herein, according to some embodiments, is a system comprising: one or more processors; memory; and one or more programs, wherein the one or more programs are stored in the memory and configured to be executed by the one or more processors, the one or more programs including instructions for carrying out any one of the above claims.

Also provided herein, according to some embodiments, is a system comprising a convolutional neural network (CNN), said CNN comprising: i) a first sub-CNN network configured to receive as input a first representation comprising a primary annotation of a test genomic mutation, wherein said primary annotation comprises a nucleotide sequence surrounding said test genomic mutation; and generate as output a first feature vector; ii) a second sub-CNN network configured to receive as input a second representation comprising at least one feature of a secondary annotation of said test genomic mutation, wherein said feature comprises a functional feature, a genomic feature, or an epigenomic feature; and generate as output a second feature vector; and iii) a third sub-CNN network configured to merge the output of the first and second sub-CNN network by receiving, as input a first feature vector generated by the first sub-CNN network, and a second feature vector generated by the second sub-CNN network; and generating as output a score indicative of a cancer risk associated with the test genomic mutation. In some embodiments, the third sub-CNN network is configured to merge the output of the first and second sub-CNN network and a third representation comprising at least one feature of said secondary annotation.

Also provided herein, according to some embodiments, is a set of probes configured to bind specifically to a plurality of oligonucleotides, each oligonucleotide comprising at least one of said plurality of cancer mutations identified by the methods described herein. In some embodiments, said plurality of oligonucleotide are cf-DNA fragments. Also provided herein, according to some embodiments, is a method of diagnosing, monitoring, or determining the state of cancer in a subject, comprising: contacting the set of probes configured to bind specifically to a plurality of oligonucleotides to a sample from a subject diagnosed with or suspected of having cancer, wherein each oligonucleotide comprises at least one of said plurality of cancer mutations identified by the methods described herein; and detecting the identity of mutations bound to the probes to diagnose, monitor, or determine the state of cancer in said subject.

### BRIEF DESCRIPTION OF THE DRAWINGS

The disclosed embodiments have other advantages and features which will be more readily apparent from the detailed description, the appended claims, and the accompanying figures (or drawings). A brief introduction of the figures is below.

FIG. 1 is a high-level block diagram illustrating the overall system environment for predicting cancer-related pathogenic impact of somatic mutations, in accordance with an embodiment.

FIG. 2A shows a visual representation of the overall process for predicting cancer-related pathogenic impact of somatic mutations, in accordance with an embodiment.

FIG. 2B illustrates the deep-learning framework for predicting cancer-related pathogenic impact of somatic mutations, in accordance with an embodiment.

FIG. 3A illustrates the system architecture of the cancer risk score determination module, in accordance with an embodiment.

FIG. 3B shows an example training data set for training the neural networks illustrated in FIG. 3A, according to an embodiment.

FIG. 3C shows a visual representation of a neural network architecture for predicting cancer-related pathogenicity of genomic mutations, in accordance with an embodiment.

FIGS. 4A and 4B show visual representations of exemplary deep convolutional neural network architecture for predicting cancer-related pathogenic impact of genomic mutations, in accordance with embodiments described herein.

FIGS. 5A and 5B illustrate embodiments of a representation of a primary annotation and sub-CNN N1 for processing via a convolutional neural network.

FIG. 6 illustrates an embodiment of a representation a secondary annotation and a sub-CNN N2 for processing via a convolutional neural network.

FIG. 7 illustrates an embodiment of formation of a merged layer from processed representations of primary and secondary annotations for a mutation, and using a sub-CNN N3 to generate a pathogenicity score for the mutation

FIG. 8 illustrates an exemplary set of representations of primary and secondary annotations for a mutation and deep convolutional neural network-based architecture for predicting cancer-related pathogenicity of genomic mutations, in accordance with an embodiment as shown in FIG. 4A.

FIG. 9A illustrates an exemplary set of representations of primary and secondary annotations for a mutation and deep convolutional neural network-based architecture for predicting cancer-related pathogenicity of genomic mutations, in accordance with an embodiment as shown in FIG. 4B. FIG. 9B illustrates an exemplary implementation of sub-CNN N1 of step 440. FIG. 9C illustrates an exemplary implementation of sub-CNN N2 and sub-CNN N3.

FIG. 10 illustrates an example process illustrating a method of using the predicted set of cancer-related somatic mutations to accurately and inexpensively detect tumor signals from blood, according to an embodiment.

FIG. 11 illustrates an example process of training and using a convolutional neural network using i) a first representation comprising a primary annotation, ii) a second representation comprising a secondary annotation, and iii) a third representation comprising a secondary annotation.

FIGS. 12A and 12B illustrate example processes for predicting cancer-related pathogenicity of genomic mutations using a convolutional neural network-based architecture, according to embodiments described herein.

FIG. 13 is a high-level block diagram illustrating an example computer for implementing the client device and/or the computer system of FIG. 1.

FIG. 14 graphs data demonstrating the sensitivity and specificity of the predictive output of the deep-learning framework described in Example 1 and illustrated in FIGS. 11A, 11B and 11C to distinguish cancer-causing somatic mutations from common mutations in two cancer types, lung cancer and breast cancer.

### DETAILED DESCRIPTION

Cancer is a genetic disease initiated by somatic mutations and progressed by an accumulation of genomic aberrations. Differentiating cancer causing mutations (i.e., driver mutations) from benign mutations (i.e., passenger mutations) not only provides valuable information for understanding molecular mechanisms of cancer, but also provides important targets for cancer diagnosis, prognosis, and disease monitoring. In order to identify cancer causing mutations and distinguish them from passenger mutations, provided herein are deep learning-based methods to predict a cancer-related pathogenicity of the test genomic mutations. In some embodiments, this cancer-related pathogenicity is in the form of a risk score. Thus, in some embodiments, the methods provided herein predict whether a genomic mutation is likely to be a cancer driver mutation or a benign mutation, such as a passenger mutation, by giving a quantitative value to predict cancer-related pathogenic risk of a given genomic mutation.

Embodiments of the invention train and/or use machine learning models, for example, deep convolutional neural networks (CNN), to predict a cancer pathogenicity of a genomic sequence variant. In some embodiments, the methods identify a cancer-associated functional impact of a sequence variant in carcinogenesis. In some embodiments, the present disclosure provides a method of predicting a cancer pathogenicity of a genomic sequence variant (i.e., a mutant). In some embodiments, the method is a computer-implemented method of predicting a cancer pathogenicity of a genomic sequence variant.

In addition, the methods provided herein can be used to determine a pathogenicity associated with any mutation. The neural networks provided herein advantageously can be used to combine two different representations of mutation-related annotations to detect an association of the mutation with a pathogenicity.

In some embodiments, provided herein is a computer-implemented method for predicting a pathogenicity of a test genomic mutation, comprising preparing primary annotation and a secondary annotation of a genomic mutation as input data and generating a score corresponding to pathogenicity of the mutation using a convolutional neural network. In some embodiments, preparing input data comprises generating a first representation comprising a primary annotation of said test genomic mutation, wherein said primary annotation comprises a nucleotide sequence surrounding said test genomic mutation, and generating a second representation comprising at least one feature of a secondary annotation of said test genomic mutation, wherein said feature comprises a functional feature, a genomic feature, or an epigenomic feature. In some embodiments, the convolutional neural network comprises a first sub-CNN network configured to process the first representation, a second sub-CNN network configured to process the second representation, and a third sub-CNN network configured to process the output of the first and second sub-CNN network. In some embodiments, generating the score corresponding to pathogenicity of the mutation comprises receiving, by the first sub-CNN network, said first representation of said test genomic mutation; generating, by the first sub-CNN network, output comprising a first feature vector; receiving, by the second sub-CNN network, said second representation of said test genomic mutation; generating, by the second sub-CNN network, output comprising a second feature vector; receiving, by the third sub-CNN network, input comprising the first feature vector and the second feature vector; and generating, by the third sub-CNN network, output representing a score indicative of a pathogenicity associated with the test genomic mutation.

Input

Dual Annotation

Mutations residing in protein-coding regions can affect protein folding and stability, protein function, and protein-protein interactions, as well as protein expression and subcellular localization. On the other hand, many disease-associated variants are located in the non-coding portions of genome and they play diverse roles in the regulation of protein-coding genes. Previous studies have shown that disease-related functional effects can be inferred from DNA sequence or protein sequence alone. However, some functional features, such as cross-species sequence conservation and distal regulatory control, are difficult to predict from sequence-only information. Therefore, as input, the machine learning model takes not only an annotation comprising genome sequence information, but also an annotation comprising a variety of functional and other features related to the sequence variant.

Given a test genomic mutation, in some embodiments, the methods provided herein first use computational tools to annotate the mutation with two sets of annotation data, a first set comprising surrounding genomic sequence context (i.e., a primary annotation), and a second set comprising one or more functional and/or genomic or epigenomic features, or relevant features related to mutation location or the flanking regions of mutation (or other relevant features) (i.e., a secondary annotation). A primary or secondary annotation can be split into one or more different representations for input into a deep-leaning framework. For example, a secondary annotation can be split into features specific to a mutation, and features corresponding to the sequence around the mutation. One or more representations of the primary and the secondary annotation is then input into a deep-learning framework to quantitatively estimate the cancer-related pathogenic impact of the test genomic mutation based on the primary and the secondary annotation representations.

Primary Annotation

In some embodiments, a primary annotation comprises the surrounding sequence (i.e., flanking sequence) of the mutation of interest. Thus, in some embodiments, the primary annotation includes a sequence of nucleotides both 5′ to and 3′ to the mutation. In some embodiments, the surrounding sequence ranges from 300 to 1,000 base pairs. In some embodiments, the sequence surrounding the mutation of interest is at least 100 bp, at least 200 bp, at least 300 bp, at least 400 bp, at least 500 bp, at least 600 bp, at least 700 bp, at least 800 bp, at least 900 bp, or at least 1000 bp. For example, a 200 bp surrounding sequence can have 100 bp 5′ of the mutation and 100 bp 3′ of the mutation.

Secondary Annotation

In some embodiments, a feature of a secondary annotation can be obtained from a database or a data repository or using computational tools based on the location or identity of the mutation. In some embodiments, a feature of a secondary annotation is derived from the mutation sequence. For example, the mutation sequence can be analyzed to determine a characteristic feature of a mutation, such as a nonsense mutation, a missense mutation, a frameshift mutation, or another feature that can be inferred from the sequence.

In some embodiments, a secondary annotation comprises a functional feature. In some embodiments, computational tools are used to annotate pathogenicity and protein-level functional impact features of mutations. In some embodiments, a secondary annotation comprises a sequence conservation feature. In some embodiments, computational tools are used to score the sequence conservation across species to generate one or more sequence conservation features.

In some embodiments, the secondary annotation comprises one or more epigenetic or epigenomic features. In some embodiments, the epigenomic features comprise histone modification, DNA methylation, transcription factor binding sites (TFBS), cis-regulatory element, regions of open chromatin, or enhancer-promoter linkage. In some embodiments, epigenomic features are obtained from data repositories (e.g., ENCODE and/or Roadmap epigenomics projects). In some embodiments, the epigenomic feature is integrated chromatin information. In some embodiments, the integrated chromatin information is obtained from computational tools (e.g., ChromHMM (Ernst, J., & Kellis, M. Nature methods. 2012) and Segway (Hoffman, Michael M., et al. Nature methods. 2012)).

In some embodiments, the secondary annotation comprises one or more genomic features. In some embodiments, the genomic feature comprises percentage of GC content, percentage of CpG islands, or repetitive sequence information. In some embodiments, the genomic feature is obtained from RepeatMasker (http://www.repeatmasker.org) and Tandom Repeats Finder (Benson, Gary. Nucleic acids research. 1999). In some embodiments, the genomic feature is determined by the machine learning system. In some embodiments, the secondary annotation comprises a gene feature. In some embodiments the gene feature is obtained from pathway information from databases (e.g., KEGG (https://www.genome.jp/kegg/)) and/or gene ontology information from the GO database (http://www.geneontology.org/).

In some embodiments, a feature of a secondary annotation is derived from the mutation sequence. For example, the mutation sequence can be analyzed to determine a characteristic feature of a mutation, such as a nonsense mutation, a missense mutation, a frameshift mutation, or another feature that can be inferred from the sequence.

In some embodiments, a feature of a secondary annotation is an epigenomic feature or an integrated epigenomic feature. For example, the integrated chromatin information is obtained from ChromHMM and Segway.

In some embodiments, a feature of a secondary annotation comprises a gene-level functional feature. For example, pathogenicity and protein-level functional impact features of mutations obtained using computational tools such as AnnoVar (described in Wang, K., et al. Nucleic acids research. 38(16): e164 (2010)), Ensembl-VEP (described in McLaren, William, et al. Genome biology, 17(1), 122 (2016)) and/or SnpEff (described in Cingolani, Pablo, et al. Fly, 6(2): 80-92 (2012)).

In some embodiments, the features are defined based on a variant type. In some embodiments, the variant type is a synonymous genetic sequence variant, a missense genetic sequence variant, a nonsense genetic sequence variant, a frame-shifting genetic sequence (such as an insertion genetic sequence variant or a deletion genetic sequence variant), a splice-site genetic sequence variant (such as a canonical splice-site genetic sequence variant or a non-canonical splice-site genetic sequence variant), a genetic sequence variant in a coding region, such as a genetic sequence variant in an intronic region, a genetic sequence variant in a promoter region, a genetic sequence variant in an enhancer region, a genetic sequence variant in a 3′-untranslated region (3′-UTR), a genetic sequence variant in a 5′-untranslated region (5′-UTR), a genetic sequence variant in an intergenic region, evolutionary conservation, regulatory element analysis, or functional genomic analysis).

In some embodiments, one or more of the features are categorical features. In some embodiments, the categorical features correspond to genetic sequence variant type. In some embodiments, the genetic sequence variant type is a synonymous genetic sequence variant, a missense genetic sequence variant, a nonsense genetic sequence variant, a frame-shifting genetic sequence variant (such as an insertion genetic sequence variant or a deletion genetic sequence variant), or a splice-site genetic sequence variant (such as a canonical splice-site genetic sequence variant or a non-canonical splice-site genetic sequence variant). In some embodiments, one or more categorical features correspond to a genomic region of the genetic sequence variant (such as a genetic sequence variant in a coding region, such as a genetic sequence variant in an intronic region, a genetic sequence variant in a promoter region, a genetic sequence variant in an enhancer region, a genetic sequence variant in a 3′-untranslated region (3′-UTR), a genetic sequence variant in a 5′-untranslated region (5′-UTR), or a genetic sequence variant in an intergenic region). These features can be classified as categorical features.

In some embodiments, one or more of the features are numerical scores, such as probability of mutation impact on protein function (e.g., SIFT scores) or evolutionary conservation (e.g., PhyloP scores or PhastCons scores).

In some embodiments, a feature that is defined on missense variants is generated using sequence homology within coding regions to determine how disruptive a missense variant in the genetic sequence variant might be. Example methods useful for generating a feature defined on missense variants include SIFT (described in Ng & Henikoff, Nucleic Acids Research, 31(13): 3812-4 (2003) and Kumar et al., Nat. Protoc. 4(7):1073-81 (2009)) and PolyPhen2 (described in Adzhubei et al., Nature Methods, 7(4):248-9 (2010)). In some embodiments, a feature that is defined on a frame-shifting genetic sequence variant is generated using sequence homology within coding regions to determine how disruptive a frame-shifting genetic sequence variant might be. Example methods useful for generating a feature defined on a frame-shifting genetic sequence variant include PROVEAN (described in Choi et al., PLoS ONE, 7(10) (2012)) and SIFT Indel (described in Hu & Ng, PLoS ONE, 8(10) (2013)). In some embodiments, the feature that is defined on missense genetic sequence variant or a frame-shifting genetic sequence variant is generated using a probabilistic model to score genetic sequence variant. Example methods useful for generating a feature defined on probabilistic scores include LRT (described in Chun & Fay, Genome Research, 19(9):1553-61 (2009)) and MAPP (described in Stone & Sidow, Genome Research, 15(7):978-86 (2005)). In some embodiments, a feature that is defined on nonsense variants is generated using sequence homology within coding regions to determine how disruptive a nonsense variant in the genetic sequence variant might be.

In some embodiments, a feature that is defined on a splice-site genetic sequence variant is generated using a predicted probability that a given genetic sequence variant will alter the splicing of a transcript. Aberrant splicing can create a large effect on a downstream protein with a very small nucleotide change, which may result in a pathogenic genetic sequence variant. Example methods useful for generating a feature defined on splice-site variants include MutPred Splice (described in Mort et al., Genome Biology, 15(1):R19 (2014)), Human Splicing Finder (HSF) (described in Desmet et al., Nucleic Acids Research, 37(9):e67 (2009)), MaxEntScan (described in Yeo & Burge, Journal of Computational Biology, 11(2-3):337-394 (2004)), and NNSplice (described in Reese et al., Journal of Computational Biology, 4(3):311-323 (1997)).

In some embodiments, a feature that is defined on a functional genomic analysis of the genetic sequence variant is generated by comparing the location and sequence of the genetic sequence variant to locations of annotated functional genomic regions. For example, in some embodiments, the functional feature evaluates the probability that a given genetic sequence variant will impact an enhancer or promoter region, or other regulatory element, in a genome. For example, the ENCODE (described in Bernstein et al., Nature, 489(7414): 57-74 (2012)) and Epigenome Roadmap (described in Kundaje et al., Nature, 518(7539):317-330 (2015)) projects, provide information about the relative functionality of different regions of the genome. Example methods useful for generating a feature defined on a functional genomic analysis of the genetic sequence variants include ChromHMM, SegWay, and FitCons (Gulko et al., Nature Genetics, 47(3):276-283 (2015)).

The methods described herein allow for annotating genetic sequence variants with an ensemble of features. In some embodiments, genetic sequence variants are annotated with 1 or more (such as 2 or more, 3 or more, 4 or more, 5 or more, 6 or more, 7 or more, 8 or more, 9 or more, 10 or more, 12 or more, 15 or more, 20 or more, 25 or more, 30 or more, 40 or more, 50 or more, or 60 or more) features. The sequences can be annotated using, for example, Ensembl-VEP. In some embodiments, a portion of the genetic sequence variants are unable to be annotated with one or more features. In some embodiments, such missing data is integrated out of the generative model. Table 1 provides examples and descriptions of features that can be used in the secondary annotation in some embodiments of the disclosed methods.

TABLE 1
Exemplary list of features that can be used for
secondary annotation of a genetic mutation

| Feature Type | Features |
|---|---|
| Basic | Chrom |
| Information | Pos |
| | Ref_Allel |
| | Alt_Allel |
| | Mutation_type |
| | Gene length |
| Genomic | chromosome_breakpoint |
| feature | clone_insert_end |
| | clone_insert_start |
| | deletion_junction |
| | exon_junction |
| | insertion_site |
| | polyA_site |
| | restriction_enzyme_cleavage_junction |
| | splice_junction |
| | trans_splice_junction |
| | CpG_island |
| | CG_content |
| | Okazaki_fragment |
| | QTL |
| | amini_acid_type |
| | epitope |
| | ligand_binding_site |
| | metal_binding_site |
| | nucleotide_binding_site |
| | protein_binding_site |
| | chromosomal_regulatory_element |
| | chromosomal_structural_element |
| | chromosome_arm |
| | chromosome_band |
| | interband |
| | introgressed_chromosome_region |
| | histone_2A_acetylation_site |
| | histone_2B_acetylation_site |
| | histone_3_acetylation_site |
| | histone_4_acetylation_site |
| | H4K_acylation_region |
| | H2BK5_monomethylation_site |
| | H3K20_trimethylation_site |
| | H3K23_dimethylation_site |
| | H3K27_methylation_site |
| | H3K36_methylation_site |
| | H3K4_methylation_site |
| | H3K79_methylation_site |
| | H3K9_methylation_site |
| | H3R2_dimethylation_site |
| | H3R2_monomethylation_site |
| | H4K20_monomethylation_site |
| | H4K4_trimethylation_site |
| | H4R3_dimethylation_site |
| | H2B_ubiquitination_site |
| | TSS_region |
| | gene_fragment |
| | pseudogenic_gene_segment |
| | intein_encoding_region |
| | non_transcribed_region |
| | recombination_regulatory_region |
| | replication_regulatory_region |
| | transcription_regulatory_region |
| | translation_regulatory_region |
| | aberrant_processed_transcript |
| | alternatively_spliced_transcript |
| | edited_transcript |
| | enzymatic_RNA |
| | mature_transcript |
| | monocistronic_transcript |
| | polycistronic_transcript |
| | predicted_transcript |
| | primary_transcript |
| | processed_transcript |
| | trans_spliced_transcript |
| | transcript_bound_by_nucleic_acid |
| | transcript_bound_by_protein |
| | transcript_with_translational_frameshift |
| | origin_of_replication |
| | polyA_sequence |
| | polypeptide |
| | polypeptide_region |
| | pseudogene |
| | pseudogenic_region |
| | rearrangement_region |
| | recombination_feature |
| | repeat_region |
| | repeat_unit |
| | replicon |
| | restriction_enzyme_region |
| | retron |
| | sequence_motif |
| | sequence_secondary_structure |
| | substitution |
| | transcript_region |
| Functional | dominant_negative_variant |
| feature | gain_of_function_variant |
| | lethal_variant |
| | loss_of_function_variant |
| | loss_of_heterozygosity |
| | null_mutation |
| | level_of_transcript_variant |
| | transcript_processing_variant |
| | transcript_stability_variant |
| | transcription_variant |
| | 3D_polypeptide_structure_variant |
| | complex_change_of_translational_product_variant |
| | polypeptide_function_variant |
| | translational_product_level_variant |
| Structural | regulatory_region_ablation |
| feature | transcript_ablation |
| | regulatory_region_amplification |
| | transcript_amplification |
| | gene_fusion |
| | regulatory_region_fusion |
| | transcript_fusion |
| | transcript_regulatory_region_fusion |
| | regulatory_region_translocation |
| | transcript_translocation |
| | feature_elongation |
| | feature_truncation |
| | gene_variant |
| | intergenic_variant |
| | regulatory_region_variant |
| | silent_mutation |
| | copy_number_change |
| | short_tandem_repeat_change |
| Gene level | cryptic_gene |
| feature | engineered_gene |
| | epigenetically_modified_gene |
| | foreign_gene |
| | fusion_gene |
| | gene_cassette |
| | gene_with_non_canonical_start_codon |
| | gene_with_polycistronic_transcript |
| | gene_with_trans_spliced_transcript |
| | mt_gene |
| | ncRNA_gene |
| | negatively_autoregulated_gene |
| | nuclear_gene |
| | nucleomorph_gene |
| | plasmid_gene |
| | plastid_gene |
| | positively_autoregulated_gene |
| | post_translationally_regulated_gene |
| | predicted_gene |
| | protein_coding_gene |
| | psudo_gene |
| | proviral_gene |
| | recombinationally_rearranged_gene |
| | rescue_gene |
| | retrogene |
| | silenced_gene |
| | transgene |
| | translationally_regulated_gene |
| | T_cell_receptor_gene |
| | immunoglobulin_gene |
| Comparative | Cons_46-Way_score |
| sequence | GERP_convervation_score |
| | Mammal_cons_score |
| | Phylop_score |
| | Grantham_score |
| | PolyPhen_score |
| | SIFT_score |
| Epigenetic | ENCODE_transcription_level |
| feature | ENCODE_H3K4me1 |
| | ENCODE_H3K4me3 |
| | ENCODE_H3K27ac |
| | ENCODE_H3K27me3 |
| | ENCODE_H3K9me3 |
| | ENCODE_H3K36me3 |
| | ENCODE_TFBS |
| | ENCODE_DNase_clusters |
| | ENCODE_Open_Chromatin |
| | Segway |
| | ChromHMM_state |
| | Roadmap_H3K4me1 |
| | Roadmap_H3K4me3 |
| | Roadmap_H3K27me3 |
| | Roadmap_H3K9me3 |
| | Roadmap_H3K36me3 |

In some embodiments, the features can be represented as a vector score or a scalar score. For example, in some embodiments a vector score is a vector of multiple levels of evolutionary conservation, such as evolutionary conservation across all vertebrates, across all mammals, or across all primates. In some embodiments, a portion of the features are vector scores. In some embodiments, a portion of the features are scalar scores. In some embodiments, one or more of the features are numerical scores, such as probability of mutation impact on protein function (e.g., SIFT scores) or evolutionary conservation (e.g., PhyloP scores or PhastCons scores).

In some embodiments, the secondary annotation is represented as a vector of numerical or categorical values, in which each value represents a genomic or epigenomic or other features from a database or a data resource or calculated from computational tools based on the location or identity of the mutation.

In some embodiments, a secondary annotation is represented as a data matrix, in which each row represents a genomic or epigenomic or other features from a database or a data resource or calculated from computational tools, and each column represents a numeric or a categorical value for a nucleotide position or a segment of nucleotide positions flanking the mutation of interest. In some embodiments, this sequence ranges from 100 to 2,000 base pairs. In some embodiments, the sequence flanking the mutation of interest is at least 100 bp, at least 200 bp, at least 300 bp, at least 400 bp, at least 500 bp, at least 600 bp, at least 700 bp, at least 800 bp, at least 900 bp, at least 1000 bp, or at 2000 bp. The segment ranges from 1 bp to 100 base pairs. In some embodiments, the sequence fragment is at least 1 bp, at least 2 bp, at least 3 bp, at least 4 bp, at least 8 bp, at least 10 bp, at least 20 bp, at least 25 bp, at least 50 bp, or at least 100 bp.

In some embodiments, the secondary annotation comprises sequence conservation along the DNA flanking sequence. For example, one or more rows of data matrix representation are used to represent evolutionary conservation, across all vertebrates, across all mammals, or across all primates. In some embodiments, one or more rows in the data matrix representation of the secondary annotation represent probability of mutation impact on protein function (e.g., SIFT scores) or evolutionary conservation (e.g., PhyloP scores or PhastCons scores or GERP++).

Training

In some embodiments, training and validation of the machine learning model is performed using training data. The training data is used both to optimize parameters of the machine learning model and to validate the machine learning model.

The training data comprises a data set of positive controls comprising known pathogenic sequence variants, and a data set of negative controls comprising known benign sequence variants. In some embodiments, the positive controls comprise known cancer driver mutations. In some embodiments, the negative controls comprise known cancer passenger mutations and common germline variants. In some embodiments the mutations in the training data sets comprise primary and secondary annotations.

In some embodiments, the data set of positive controls and/or the data set of negative controls comprise 100 or more genetic sequence variants, 200 or more genetic sequence variants, 300 or more genetic sequence variants, 500 or more genetic sequence variants, 750 or more genetic sequence variants, 1,000 or more genetic sequence variants, 1,250 or more genetic sequence variants, 1,500 or more genetic sequence variants, or 2,000 or more genetic sequence variants.

A data set with known cancer driver mutations can be obtained for example by filtering variants from COSMIC (described in Forbes, Simon A., et al. Nucleic acids research, 43.D1: D805-811 (2014)), ClinVar (described in Landrum, Melissa J., et al. Nucleic acids research, 42.D1: D980-5 (2013)), or Emory Genetics Laboratory (EmVClass, http://www.egl-eurofins.com/emvclass/emvclass.php) databases. The data set with known benign mutations can be obtained, for example, by filtering variants from the 1000 Genomes Project (1000G) (described in Abecasis et al., Nature, 491(7422):56-65 (2012)) and dbSNP database (https://www.ncbi.nlm.nih.gov/projects/SNP/).

Use of a Trained Machine Learning Model

Once a machine learning model has been trained using datasets with pathogenic and benign mutations with primary and secondary annotations, it can then be used to predict a cancer-related pathogenicity of the test genomic mutations. In some embodiments, the machine learning model can be used to predict a cancer-related pathogenicity of a mutation or a series of mutations artificially generated from a base genomic sequence. In some embodiments, an entire genome can be assayed to predict a cancer-related pathogenicity for all possible mutations. In some embodiments, the prediction can be limited to coding regions or to regulatory regions of genomic DNA. The risk score can be specific to cancer type or tissue type. The risk score can also be specific to other characteristics of a patient, such as age, sex, or ethnicity.

In some embodiments, the machine learning model can be used to predict a cancer-related pathogenicity from a set of mutations from a patient. These can be used to diagnose a patient or monitor the progression of disease. In some embodiments, the machine learning model identifies at least one sequence variant that is previously not known or expected to be causative of disease, but is found to exist in people with a particular disease or disorder.

In some embodiments, the trained machine learning model as described herein is applied to a test genetic sequence variant to obtain an output score. The output score is a predicted probability that the test genetic sequence variant is pathogenic.

In some embodiments, the machine learning model assigns the test genetic sequence variant to a benign cluster or a pathogenic cluster. In some embodiments, the benign cluster comprises a plurality of benign sub-clusters. In some embodiments, the pathogenic cluster comprises a plurality of pathogenic sub-clusters. In some embodiments, the test genetic sequence variant is a human genetic sequence variant.

In some embodiments, a probe set that specifically binds to a subset of driver mutations identified by the machine learning model can be used to identify whether one or more of the mutations are present in the genomic DNA of the patient, thereby assessing a cancer state for the patient. In some embodiments, these probes target cfDNA.

Any assay known in the art may be used to determine presence or absence of a genetic variation. Conventional methods can be used, such as those employed to make and use nucleic acid arrays, amplification primers, hybridization probes.

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures.

Overall System Environment

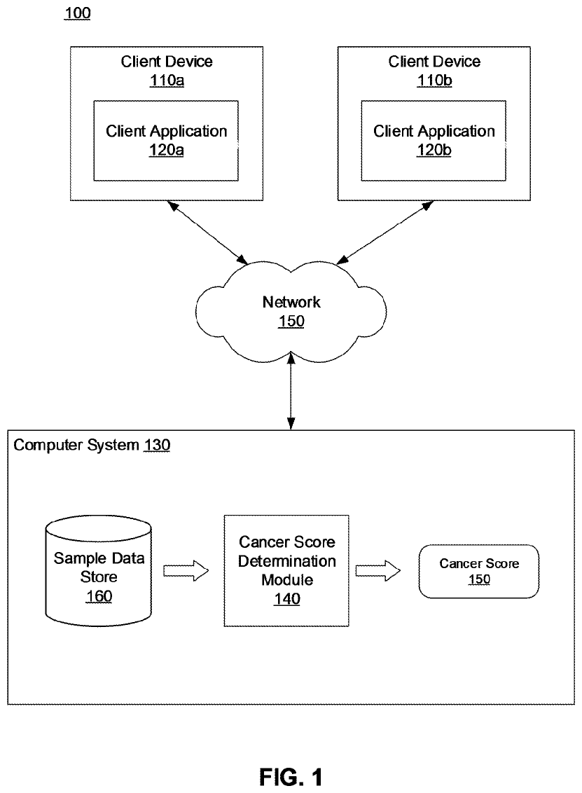

FIG. 1 is a high-level block diagram illustrating the overall system environment for predicting a cancer-related pathogenicity of a genomic mutation, in accordance with an embodiment. The system environment 100 includes one or more client devices 110 connected by a network 150 to a computer system 130. The computer system 130 includes a deep-learning framework to predict cancer-related pathogenic risk of the test somatic mutations in carcinogenesis. The client devices 110 execute client applications that allow a user to interact with the computer system 130.

Here only two client devices 110a, 110b are illustrated but there may be multiple instances of each of these entities. For example, there may be several computer systems 130 and dozens or hundreds of client devices 110 in communication with each computer system 130. The figures use like reference numerals to identify like elements. A letter after a reference numeral, such as “110a,” indicates that the text refers specifically to the element having that particular reference numeral. A reference numeral in the text without a following letter, such as “110,” refers to any or all of the elements in the figures bearing that reference numeral.

The network 150 provides a communication infrastructure between the client devices 110 and the record management system 130. The network 150 is typically the Internet, but may be any network, including but not limited to a Local Area Network (LAN), a Metropolitan Area Network (MAN), a Wide Area Network (WAN), a mobile wired or wireless network, a private network, or a virtual private network. Portions of the network 150 may be provided by links using communications technologies including WiFi based on the IEEE 802.11 standard, the BLUETOOTH short range standard, and the Wireless Universal Serial Bus (USB) standard.

The client devices 110 are computing devices such as smartphones with an operating system such as ANDROID® or APPLE® IOS®, tablet computers, laptop computers, desktop computers, electronic stereos in automobiles or other vehicles, or any other type of network-enabled device on which digital content may be listened to or otherwise experienced. Typical client devices 110 include the hardware and software needed to connect to the network 150 (e.g., via Wifi and/or 4G or other wireless telecommunication standards).

The client device 110 includes a client application 120 that allows a user of the client device 110 to interact with the computer system 130. For example, the client application 120 may display a user interface that allows a user to enter data including the training data and inputs provided to the trained model and display results of execution of the model. In an embodiment, the client application 120 is a browser that allows users of client devices 110 to interact with the computer system 130 by interacting with websites, for example, a website that allows users to check-in software.

The computer system 130 includes software for performing a group of coordinated functions or tasks. The software may allow users of the computer system 130 to perform certain tasks or activities of interest, or may include system software (e.g., operating systems) that provide certain functionalities and services to other software. The computer system 130 receives requests from client devices 110 and executes computer programs associated with the received requests. As an example, the computer system 130 may execute computer programs responsive to a request from a client device 110 to test updates to various software components of the computer system 130. The computer system 130 is also referred to herein as the system. In some embodiments, the client application executes on the computer system 130 such that a user interacts with the cancer score determination module 140 using the peripheral devices of the computer system 130 rather than the client device 110.

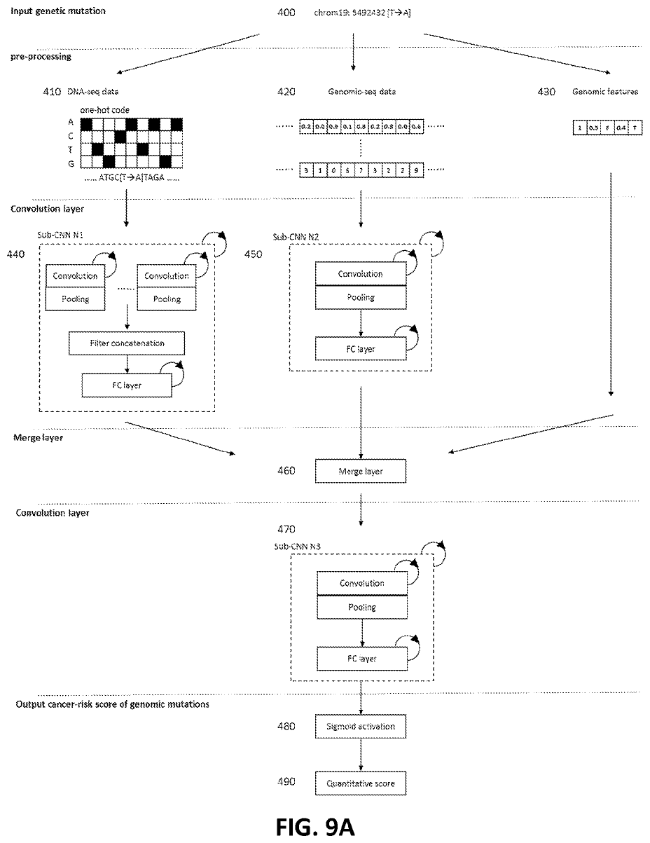

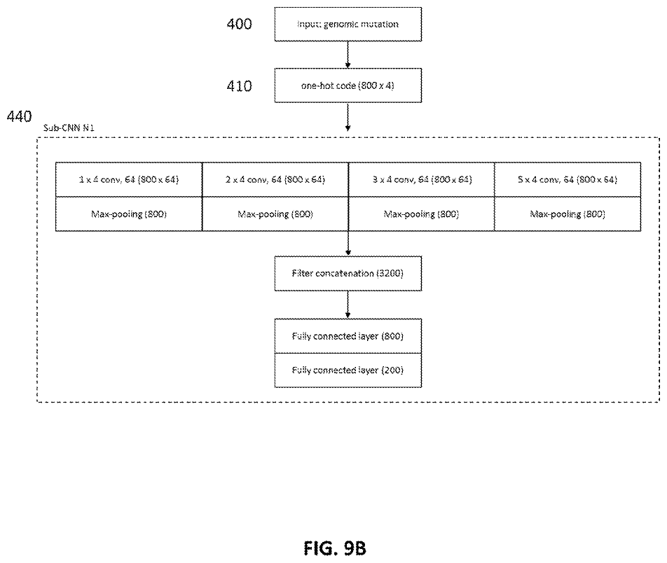

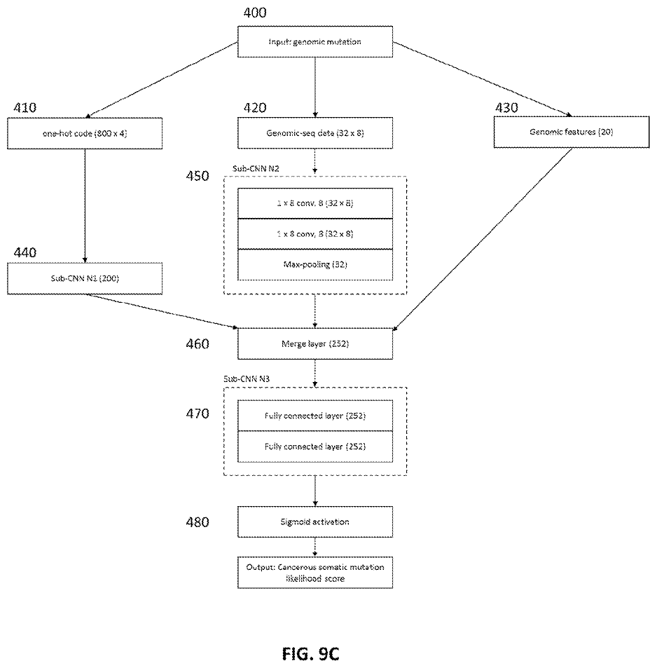

The disclosed methods are highly computation intensive and therefore, a computer system 130 used to process the disclosed methods is typically a powerful machine. The input of an example model comprises: (1) 100,000 of 4×800 numerical data matrices, where 100,000 represents the total number of mutations for test (both positive and control datasets); and 4×800 represents the size of a one-hot-code encoded numerical data matrix to represent 800 bp flanking sequence of each mutation. (2) 100,000 of 8×32 numerical data matrices, wherein the 8×32 numerical data matrix is for 8 genetic and epigenetic features for 32 non-overlapping 25 bp DNA segments (corresponding to 32×25=800 bp flanking sequence) for each mutation. (3) 100,000 of 20-dimensional vector, where each 20-dimensional vector is used to represent a variety of functional features related to the test mutation or mutation-associated sequence segment. The example model contains 2.86M parameters in total for learning (see, e.g., FIG. 9A-9C for additional details).

An example computer system 130 used to train the example model is graphical processor unit (GPU) based and includes 16 NVIDIA K80 GPUs, 64 CPUs and 732 GB of host memory, with a combined 192 GB of GPU memory, 40 thousand parallel processing cores, 70 teraflops of single precision floating point performance, and over 23 teraflops of double precision floating point performance. Using a computer system 130 with such a configuration, the training of the example model takes about 20 hours with approximately 2000 epochs. Furthermore, typical executions of the training process comprise multiple training runs to validate the resultant model and to avoid potential local-minima problems.

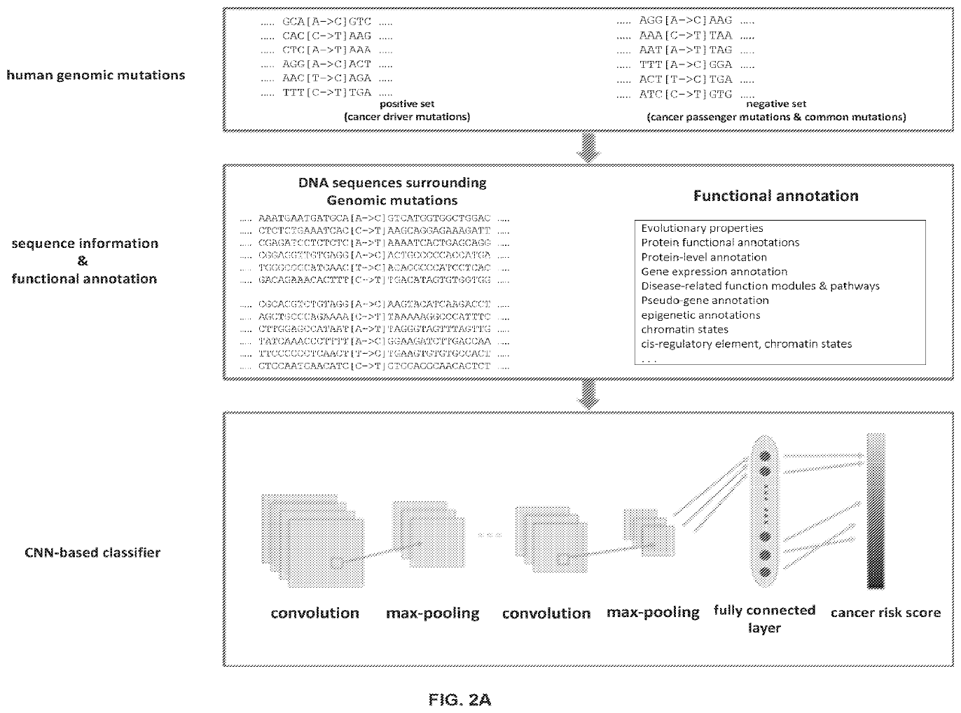

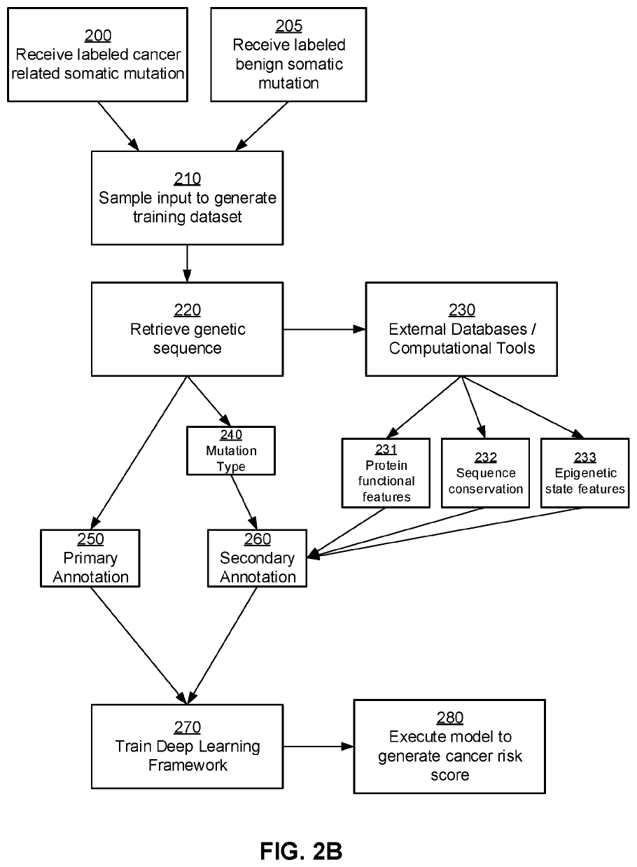

FIG. 2A shows a visual representation of the overall process for predicting cancer-related pathogenic impact of somatic mutations, in accordance with an embodiment. In the first step, the computer system 130 constructs a positive dataset of known cancer driver mutations and a control dataset containing cancer passenger mutations and common germline variants. In an embodiment, the cancer-associated mutation dataset is built from three public cancer sources, COSMIC, ClinVar, and Emory Genetics Laboratory (EmVClass). In detail, the mutations labelled as “confirmed” in COSMIC and “pathogenic or likely pathogenic in cancer” in ClinVar and EmVClass are categorized as high-confident cancer driver mutations and are included in the positive dataset. The control dataset contains two types of mutations: the common germline variants from 1000 Genomes Project and dbSNP database, and the somatic mutations labelled as “benign or likely benign” in ClinVar and EmVClass. In some embodiments, the control dataset comprises benign germline variants and benign somatic variants. In some embodiments, a subset is down-sampled from the control dataset to keep two datasets balanced. In some embodiments, the positive dataset is over-sampled to from the positive dataset to keep two datasets balanced. In some embodiments, down-sampling and over-sampling are performed on control data and positive dataset respectively, to keep two datasets balanced.

In the second step, the system retrieves DNA sequence context around each mutation from the human reference genome to generate a primary annotation, and generates a secondary annotation comprising features relevant to each mutation that is not the flanking sequence using a diversity of tools. In some embodiments, the secondary annotation comprises a functional feature, a sequence conservation feature, an epigenetic feature, an integrated chromatin feature, a genomic feature, a gene-level feature, or any combination thereof.

In an embodiment, for secondary annotation, the system uses functional feature tools, for example, AnnoVar, Ensembl-VEP or SnpEff to identify pathogenicity and protein-level functional impact features of mutations. Further, in some embodiments, the system uses phastCons, phyloP and GERP++ to score the sequence conservation across species to determine a sequence conservation feature. An epigenetic feature, including histone modification, DNA methylation, transcription factor binding sites (TFBS), cis-regulatory element, regions of open chromatin, enhancer-promoter linkage, can be obtained from ENCODE and Roadmap epigenomics projects. In some embodiments, the system extracts integrated chromatin features from ChromHMM and Segway. In some embodiments, the system calculates a genomic feature, such as percentage of GC content, percentage of CpG islands, repetitive sequence information from RepeatMasker and Tandom Repeats Finder. In some embodiments, a gene-level feature is obtained from pathway information from KEGG or gene ontology information from the GO database.

Values for features that are used by the machine learning system but have no observable value for a given mutation may be recorded in a number of ways. In some cases, missing values of specific features within the secondary annotation may contain a default value. In some embodiments, missing values of specific features within the secondary annotation can be assigned a default value. In some embodiments, missing values of specific features within the secondary annotation are imputed from the surrounding genomic regions (e.g., taking the average of the values from the nearby genomic region). In some embodiments, highly correlated features are reduced by examining correlations between features and the value of adding interaction terms between features.

In the third step, the system uses the primary and secondary annotation data from a positive control data set and a negative control data set to train a machine learning based model, for example, a deep convolutional neural network to distinguish cancer driver mutations from passenger mutations and germline common variants. The basic layers in the model are convolution layers and max-pooling layers. By jointly learning a local connectivity pattern from adjacent neurons, the CNN model extracts spatially-local correlation features and passes such information to the higher layers. This information flow architecture ensures that the CNN model efficiently detects the learnt features and produces strong response to spatial patterns at multiscale. A good example is utilizing CNN models to recognize objects from collections of images: the early layers of CNN models learn local features (i.e., edges) from spatially connected image pieces, and the later layers jointly learn these local features to see the “big picture” for object recognition. Similarly, the CNN model illustrated in FIG. 2B comprises early layers that learn local features and later layers that combine the local features to generate higher level features. Since the input of the model contains two different types of data: e.g., sequential data (i.e., DNA sequence data, sequence conservation score at nucleotide level) contained in a primary annotation and sparse mutation-level or gene-level functional features contained in a secondary annotation, the system uses two separate sub-CNN models to learn features of each type of input data, and then merges these two subnetworks to jointly learn features and calculate cancer risk scores.

FIG. 2B illustrates the deep-learning framework for predicting cancer-related pathogenic impact of somatic mutations, in accordance with an embodiment. The system receives input data comprising labeled cancer related somatic mutations 200 as positive control examples and labeled benign somatic mutations 205 as negative control examples. The system performs sampling of these inputs to generate the training dataset 210. The system retrieves a genetic sequence for each sample comprising the mutation and flanking nucleotide sequence 220. The system prepares the input for providing to the deep learning framework to train the model.

The genetic sequence can be used directly as a primary annotation 250 to be input into the deep learning framework 270 for training. The genetic sequence can also be processed to determine a feature of the mutation related to the mutation type 240. For example, the sequence can be processed to determine whether the mutation is a nonsense, a missense mutation, or a codon shift mutation. This feature developed from processing the sequence can then be added to a secondary annotation 260. The sequence can also be used to identify features of the mutation from an external database or using computational tools 230. These features can include, in some embodiments, protein functional features 231, sequence conservation features 232, and/or epigenetic state features 233. Other features that can added to the secondary annotation are described herein. The secondary annotation 260 comprising one or more features can be input into the deep learning framework 270 for training.

The deep learning framework 270 processes both a primary annotation 250 and a secondary annotation 260 to generate a cancer risk score 280. The trained model can be executed 280 generate cancer risk scores for input samples comprising specific mutations. These samples can also be validation samples, or unknown samples.

System Architecture

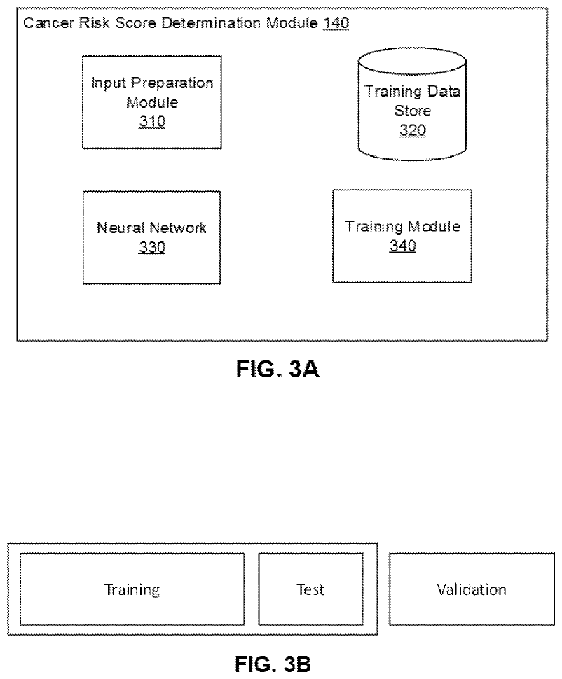

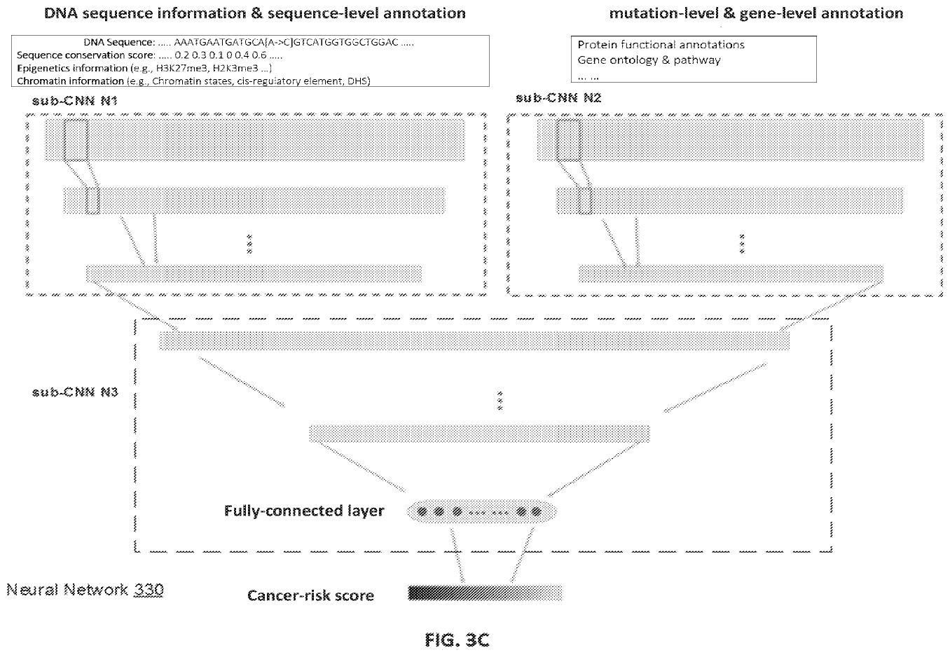

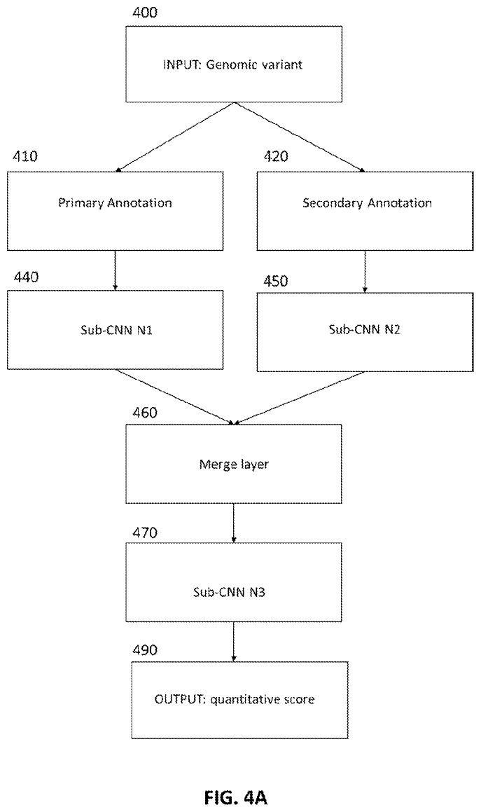

FIG. 3A illustrates the system architecture of the cancer risk score determination module, in accordance with an embodiment. The cancer risk score determination module 140 comprises an input preparation module 310, a neural network 330, a training module 340, and a training data store 320. In other embodiments, the cancer risk score determination module 140 may include additional, fewer, or different components for various applications. Conventional components such as network interfaces, security functions, load balancers, failover servers, management and network operation consoles, and the like are not shown so as to not obscure the details of the system architecture.

The neural network 330 is a machine-learned neural network model with layers of nodes, in which values at nodes of a current layer are a transformation of values at nodes of a previous layer. The transformation is determined through a set of weights connecting the current layer and the previous layer. Details of the neural network 330 are further described in connection with FIG. 3C through FIG. 11C.

The input preparation module 310 encodes input data to a form that is provided as input to the neural network 330. The input preparation module 310 receives a description of a genomic mutation and generates vector representations based on the genomic mutation that are provided as input to the neural network 330. The input preparation module 310 generates the input representations for the neural network 330 during training of the neural network 330 as well as during execution of a trained neural network 330. In an embodiment, the input preparation module 310 generates two vectors for providing as input to the neural network 330, a first vector representing a primary annotation (e.g., the genomic sequence data flanking the mutation) and a second vector representing a secondary annotation (e.g., other features relevant to the mutation, such as functional, genomic, and structural information). Further details of the input representations generated by the input preparation module 310 are described in connection with FIG. 3C through FIG. 11C.

The training module 340 uses training datasets stored in the training data store 320 to train the neural network 330. During the training process, the training module 340 determines weights associated with edges of the neural network 330. The training module 340 trains a neural network (e.g., the neural network shown in FIG. 3C).

In the neural network 330, nodes are connected together to form a network. The node characteristics values may be any values or parameters associated with a node of the neural network. The nodes may represent input, intermediate, and output data. In an embodiment, a neural network 330 includes an input layer, one or more hidden layers, and an output layer. Nodes of the input layer are input nodes, nodes of the output layer are output nodes, and nodes of the hidden layers are hidden nodes. Nodes of a layer may provide input to another layer and may receive input from another layer. Nodes of each hidden layer are associated with two layers, a previous layer and a next layer. The hidden layer receives the output of the previous layer as input and provides the output generated by the hidden layer as input to the next layer.

Each node has an input and an output. Each node of the neural network is associated with a set of instructions corresponding to the computation performed by the node. The set of instructions corresponding to the nodes of the neural network may be executed by one or more computer processors.

Each connection between the nodes (e.g., network characteristics) may be represented by a weight (e.g., numerical parameter determined in a training/learning process). In some embodiments, the connection between two nodes is a network characteristic. The weight of the connection may represent the strength of the connection. In some embodiments, a node of one level may only connect to one or more nodes in an adjacent hierarchy grouping level. In some embodiments, network characteristics include the weights of the connection between nodes of the neural network. The network characteristics may be any values or parameters associated with connections of nodes of the neural network. Details of the neural network 330 are illustrated in and described in connection with FIG. 3C through 11C.

In each training step, the training module 340 adjusts values for weights of the neural network 330 to minimize or reduce a loss function between outputs generated by propagating encoded vectors and test cases through the neural network model. Specifically, the loss function indicates difference between the labelled output corresponding to an input and a predicted output value. Minimizing the loss function aims to generate outputs that are close to the labels of corresponding to the inputs.

The training data store 320 stores various training datasets for training the convolutional neural network 330. A training dataset is composed of two datasets: a positive dataset with confirmed cancer driver somatic mutations, and a control dataset consisting of benign somatic mutations or common germline variants. The cancer driver mutation dataset could be built from public cancer data resources, i.e., COSMIC, ClinVar, or Emory Genetics Laboratory (EmVClass). In detail, the mutations labeled as “confirmed” in COSMIC and “pathogenic or likely pathogenic in cancer” in ClinVar and EmVClass can be categorized as high-confident cancer driver mutations to be included the positive dataset. The control dataset can consist of two types of genetic variations: the common germline variants (from 1000 Genomes Project, EVS, ExAC, dbSNP database), and the benign somatic mutations labelled as “benign or likely benign” in ClinVar and EmVClass datasets. The control dataset can also be generated from simulating of genomic mutations using an evolutionary model, for example, the General Time Reversible (GTR) model as described in Nature Genetics 46, 310-315 (2014), incorporated herein by reference.

In order to reduce the training biases, we could sample from the two datasets to generate a positive dataset and a control dataset with equal number of mutations. In addition, we could sample the mutations from a selected set of genomic regions, for example oncogenes, to potentially improve model accuracy.

FIG. 3B shows an example training data set for training the neural networks illustrated in FIG. 2A and FIG. 2B, according to an embodiment. In some embodiments, cross-validation is used during the training process of machine learning model to reduce overfitting. In some embodiments, for CNN models, similar approaches such as dropout and early stopping are used to reduce overfitting. As shown in FIG. 3B, the datasets can be split into two segments, one for training and the other one for model validation. In some embodiments, mini-batches are used to reduce overfitting.

Neural Network Architecture

A deep convolutional neural network (CNN) is a type of multilayer neural network organized by a sequential layer-by-layer stages to execute a sequence of function transformations. Each convolutional layer is typically composed of a number of computational units called neurons, with learnable weights and biases. Each neuron receives input from either the previous layer or the initial input and outputs a single value. Layers that are not in the input or output layer are conventionally called hidden layers, and a deep neural network indicates a neural network with more than one hidden layer. The depth of a deep neural network corresponds to the number of hidden layers, and the width indicates the maximum number of neurons in a layer.

In some embodiments, the CNN model is trained using the standard back-propagation algorithm. In some embodiments, mini-batch gradient descent with AdaGrad optimization or an alternative technique is used to improve convergence performance, and dropout and early stopping is used to reduce overfitting.

Although the embodiments described herein are based on convolutional neural networks, the techniques disclosed herein can be implemented using other types of neural networks, for example, recursive neural networks (RNN) or multi-layer perceptrons. Furthermore, each sub-neural network can be a different type of neural network.

In an embodiment, cancer risk score determination model 140 can use any machine learning model M instead of neural network 330. The machine learning model M comprises a sub-model M1, a sub-model M2, and a sub-model M3. The sub-model M1 receives the representation of the primary annotation of the input genomic mutation as input. The sub-model M2 receives the representation of the secondary annotation of the input genomic mutation as input. Both sub-model M1 and sub-model M2 process their respective inputs to generate feature vectors that are provided as input to the sub-model M3. The sub-model M3 processes the feature vectors to generate the score indicative of a cancer risk associated with the input genomic mutation. Each sub-model can be either a machine learning based model or a neural network. In one embodiment, the sub-model M1 is a CNN and the sub-model M2 is a regression-based machine learning model. In another embodiment, the sub-model M1 is a CNN and sub-model M2 is a perceptron.

FIG. 3C shows a visual representation of an example deep convolutional neural network architecture for predicting cancer-related pathogenic impact of genomic mutations, in accordance with an embodiment. The neural network 330 receives a representation of a genomic mutation and processes it to generate a score indicative of a cancer risk associated with the input genomic mutation. The neural network 330 comprises a sub-CNN N1, a sub-CNN N2, and sub-CNN N3, each sub-CNN comprising multiple layers of nodes. The sub-CNN N1 receives as input, a representation of a primary annotation of the input genomic mutation comprising a nucleotide sequence flanking the genomic mutation. The sub-CNN N2 receives as input, a representation of a secondary annotation of the input genomic mutation comprising functional features or genomic features. Both sub-CNN N1 and sub-CNN N2 process their respective inputs to generate feature vectors that are provided as input to the sub-CNN N3. The sub-CNN N3 processes the feature vectors to generate the score indicative of a cancer risk associated with the input genomic mutation. In an embodiment, the last layer of the sub-CNN N3 is a fully connected layer, in which all neurons are connected and overall predict scores are calculated. These scores are further transformed to the 0-1 range using the sigmoid function (sigmoid function: 1/(1+exp(−x))).

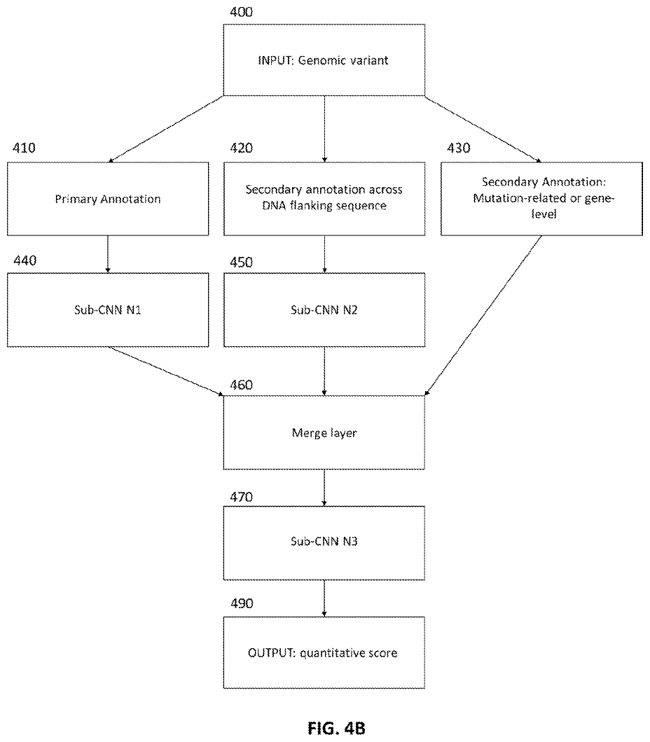

FIG. 4A shows a visual representation of an example deep convolutional neural network architecture for predicting cancer-related pathogenic impact of genomic mutations, in accordance with an embodiment. As shown in FIG. 4A, the CNN network includes three sub-CNN components: sub-CNN N1, sub-CNN N2, and sub-CNN N3. Receiving a test mutation as of step 400 as input, the convolutional neural network is constructed and trained, and outputs a quantitative score as in 490. In some embodiments, the quantitative score ranging from 0 to 1, representing a pathogenic risk (such as a cancer risk) score corresponding to the input sample.

Given a genomic variant (400), the primary annotation of DNA flanking sequence around variant is encoded (410). The secondary annotation information is retrieved from a variety of databases and data resources and calculated from computational tools. The sub-CNN N1 (440) is a CNN module to analyze the encoded DNA flanking data from 410. The sub-CNN N2 (450) is a CNN module to process secondary annotation information from 420. The merge layer (460) is used to merge the processed data from the modules comprising sub-CNN N1 (440) and sub-CNN N2 (450). The sub-CNN N3 (470) is a CNN module to analyze the merged data from 460. From the output of sub-CNN N3 (470), a quantitative score (490) can be determined associated with the pathogenicity of one or more mutations. In an embodiment, a softmax activation function (e.g., sigmoid function: 1/(1+exp(−x))) is used to transform the output of 470 into a quantitative score ranging from 0 to 1.

In some embodiments, one or more sub-CNNs can be part of a module comprising other processing components, including parallel sub-CNNs.

FIG. 4B shows a visual representation of an example deep convolutional neural network architecture for predicting cancer-related pathogenic impact of genomic mutations, in accordance with an embodiment. As shown in FIG. 4B, the CNN network includes three sub-CNN components: sub-CNN N1, sub-CNN N2, and sub-CNN N3. Receiving a test mutation as of step 400 as input, the convolutional neural network is constructed and trained, and outputs a quantitative score as in step 490. In some embodiments, the quantitative score ranges from 0 to 1, representing a pathogenic risk (such as a cancer risk) score corresponding to the input sample.

Given a genomic variant (400), the primary annotation of DNA flanking sequence around variant is encoded (410). The secondary annotation information is retrieved from a variety of databases and data resources and calculated from computational tools. Then output data is separated according to data format as shown in 420 and 430. The sub-CNN N1 (440) is a CNN module to analyze the encoded DNA flanking data from 410. The sub-CNN N2 (450) is a CNN module to process secondary annotation information from 420. Moreover, mutation-related or gene-level annotation information of the secondary annotation are represented as a scalar of values in 430. The merge layer (460) is used to merge the processed data from three modules of 440,450, and 430. The sub-CNN N3 (470) is a CNN module to analyze the merged data from 460. From the output of sub-CNN N3 (470), a quantitative score (490) can be determined associated with the pathogenicity of one or more mutations. In an embodiment, a softmax activation function (e.g., sigmoid function: 1/(1+exp(−x))) is used to transform the output of 470 into a quantitative score ranging from 0 to 1.

In some embodiments, the primary annotation is sequence data of a linear segment of genomic sequence from the human reference genome centered at the given genomic mutation. Initially, using the one-hot code, the sequence data can be encoded to a 4×L matrix, where L denotes the length of the sequence (e.g., 800 bp) and each column is a 4-element vector (e.g., [1, 0, 0, 0], [0, 1, 0, 0], [0, 0, 1, 0], [0, 0, 0, 1]) to indicate the presence or absence of A, C, G, or T at each nucleotide position.

In some embodiments, IUPAC code are used to encode ambiguous nucleotides. In detail, R (=G, A), Y (=T, C), M (=A, C), S(=G, C), W(=A, T), B(=G, T, C), D(=G, A, T), H(=A, C, T), V(G, C, A), N(A, C, G, T) are encoded as [1, 0, 1, 0], [0, 1, 0, 1], [1, 1, 0, 0], [0, 1, 1, 0], [1, 0, 0, 1], [0, 1, 1, 1], [1, 0, 1, 1], [1, 1, 0, 1], [1, 1, 1, 0], [1, 1, 1, 1], respectively.

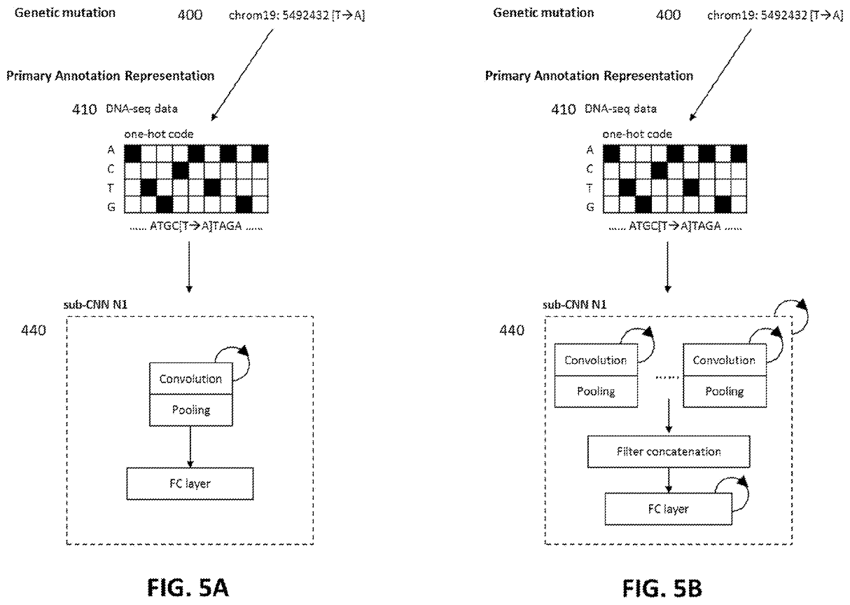

The sub-CNN N1 is a CNN module to analyze DNA flanking sequence information around the test mutation. Two examples are given in FIGS. 5A and 5B.

In FIG. 5A, sub-CNN N1 (440) is composed of one or more convolutional filters (alternating convolutional layers and nonlinear pooling layers), followed by a full-connected layer (FC layer) in the end. In detail, at each convolutional layer, n filters with window size t are applied to convolute the input from the previous layer, where n is the number of the filter and t is the window size for scanning. The weights of each convolutional layer are parameters learned from the data. After convolving the matrix across the sequence, a rectified linear unit (ReLU) (f(x)=max(0,x)) can be applied to the output to avoid the vanishing gradient problem. Furthermore, a pooling layer is applied to reduce the number of parameters and achieve invariance due to small sequence shifting. For DNA sequences in the protein-coding region, these filters can be considered as detectors to learn various protein-coding features (e.g., codons, linkage disequilibrium, and biophysical or biochemical features) from genomic sequence at different spatial resolution. For DNA sequences in noncoding region, these filters can be considered as position weight matrices (PWMs) to search for motifs along the sequence to detect regulatory elements. After one or more iterations, one or more fully connected layers are applied to the model.

FIG. 5B illustrates another example of sub-CNN N1. In this example, sub-CNN N1 contains a number of convolutional filters with different window size in parallel, followed by a FC layer in the end. These convolutional filters are of different with size to learn from DNA flanking sequence at different spatial resolution. For instance, a convolution filter with window size 1 corresponds to learn from DNA sequence at single-nucleotide resolution; a convolutional filter with window size 2 indicates dinucleotide resolution; a convolutional filter with window size 3 indicates triple-nucleotide resolution or amino-acid resolution; a convolutional filter with window size 8 indicates 8mer resolution, where 8mer is the typical length of many DNA motifs. These convolution filters are further concatenated using a filter concatenation layer, followed by a FC layer to learn genetic correlative relationships among DNA flanking sequence of the test mutation.

In some embodiments, the secondary annotation, which is composed of annotation information across DNA flanking sequence of the test mutation, is represented as a data matrix as of step 420. In such a data matrix, each row denotes an annotated feature over the DNA flanking sequence, and each column denotes different annotated features at a nucleotide position or over a DNA segment. For example, a quantitative score reflects cross-species sequence conservation at a specific nucleotide, or a percentage number indicates the GC percentage of a 25 bp DNA segment.

In some embodiments, the secondary annotation is represented as a scalar of numerical values as of step 430, of which each numeric value indicates a specific annotated feature of the test genetic variation.

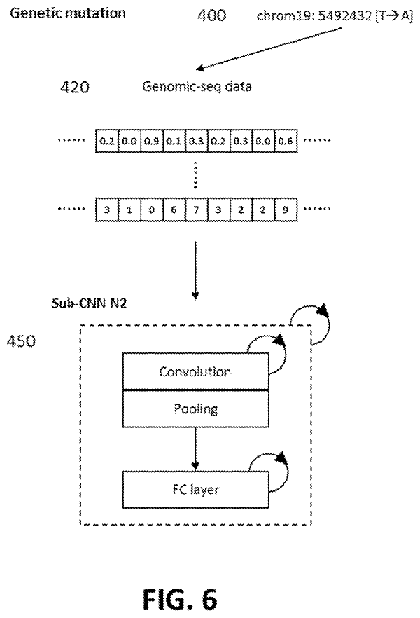

FIG. 6 illustrates an example of sub-CNN N2. Similar as sub-CNN N1 example in FIG. 5A, sub-CNN N2 is composed of one or more convolutional filters (alternating convolutional layers and nonlinear pooling layers), followed by a fully connected (FC) layer in the end.

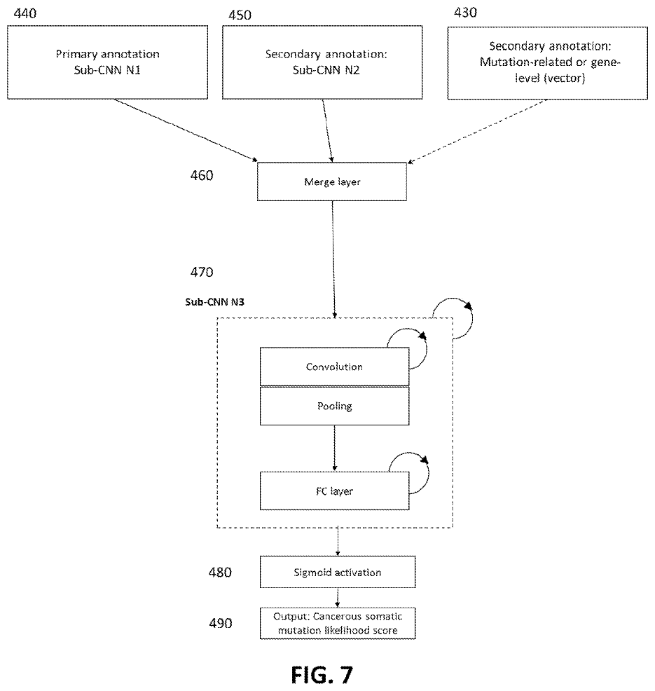

Shown in FIG. 7 is an embodiment of formation of a merged layer from processed representations of primary and secondary annotations for a mutation, and using a sub-CNN N3 to generate a pathogenicity score for the mutation. Step 460 illustrates a merge layer to merge the list of output from steps 440, 450, and 430. In detail, the merge layer concatenates the output data matrices or vectors from steps 440, 450, and 430 to a single data matrix or vector.

An example sub-CNN N3 is shown in FIG. 7 at step 470. Similar as sub-CNN N1 (example in FIGS. 5A & 5B) and sub-CNN N2 (example in FIG. 6), sub-CNN N3 can be composed of convolutional layers, followed by one or more FC layers.

In step 480, a non-linear transformation with sigmoid activation is used to transform the outputs of step 470 to a single value between 0 and 1, reflecting the pathogenic risk 490 of the give mutation.

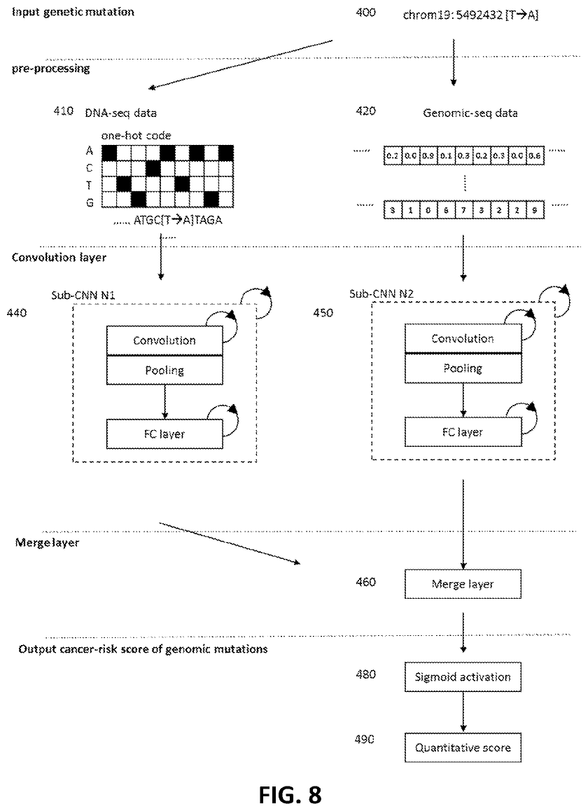

FIG. 8 illustrates an exemplary deep convolutional neural network-based architecture for predicting pathogenicity of genomic mutations, in accordance with an embodiment. Given a test genomic mutation, the input of the network includes two types of annotations: DNA flanking sequence of the test mutation (primary annotation) as of step 410; and genetic or epigenetic or other features over the DNA flanking sequence of the test mutation (secondary annotation) as of step 420.

In some embodiments, the primary annotation is sequence data of a linear segment of genomic sequence from the human reference genome centered at the given genomic mutation. Initially, using the one-hot code, the sequence data can be encoded to a 4×L matrix, where L denotes the length of the sequence (e.g., 800 bp) and each column is a 4-element vector (e.g., [1, 0, 0, 0], [0, 1, 0, 0], [0, 0, 1, 0], [0, 0, 0, 1]) to indicate the presence or absence of A, C, G, or T at each nucleotide position.

In some embodiments, IUPAC code are used to encode ambiguous nucleotides. In detail, R (=G, A), Y (=T, C), M (=A, C), S(=G, C), W(=A, T), B(=G, T, C), D(=G, A, T), H(=A, C, T), V(G, C, A), N(A, C, G, T) are encoded as [1, 0, 1, 0], [0, 1, 0, 1], [1, 1, 0, 0], [0, 1, 1, 0], [1, 0, 0, 1], [0, 1, 1, 1], [1, 0, 1, 1], [1, 1, 0, 1], [1, 1, 1, 0], [1, 1, 1, 1], respectively.

In some embodiments, the secondary annotation, which is composed of annotation information across DNA flanking sequence of the test mutation, is represented as a data matrix as of step 420. In such a data matrix, each row denotes an annotated feature over the DNA flanking sequence, and each column denotes different annotated features at a nucleotide position or over a DNA segment. For example, a quantitative score reflects cross-species sequence conservation at a specific nucleotide, or a percentage number indicates the GC percentage of a 25 bp DNA segment.

In step 440, sub-CNN N1 is composed of one or more convolutional filters (alternating convolutional layers and nonlinear pooling layers), followed by a full-connected layer (FC layer) in the end. In detail, at each convolutional layer, n filters with window size t are applied to convolute the input from the previous layer, where n is the number of the filter and t is the window size for scanning. The weights of each convolutional layer are parameters learned from the data. After convolving the matrix across the sequence, a rectified linear unit (ReLU) (f(x)=max(0,x)) can be applied to the output to avoid the vanishing gradient problem. Furthermore, a pooling layer is applied to reduce the number of parameters and achieve invariance due to small sequence shifting. For DNA sequences in the protein-coding region, these filters can be considered as detectors to learn various protein-coding features (e.g., codons, linkage disequilibrium, and biophysical or biochemical features) from genomic sequence at different spatial resolution. For DNA sequences in noncoding region, these filters can be considered as position weight matrices (PWMs) to search for motifs along the sequence to detect regulatory elements. After one or more iterations, one or more fully connected layers are applied to the model.

Step 450 illustrates an example of sub-CNN N2. Similar as sub-CNN N1 of step 410, sub-CNN N2 is composed of one or more convolutional filters (alternating convolutional layers and nonlinear pooling layers), followed by a fully connected (FC) layer in the end.

Step 460 illustrates a merge layer to concatenate the resultant matrices or vectors from steps 440 and 450 to a single data matrix or vector.

In step 480, a non-linear transformation with sigmoid activation is used to transform the outputs from the two CNN models to a single value between 0 and 1, reflecting the pathogenic risk 490 of the give mutation.

FIG. 9A illustrates an exemplary deep convolutional neural network-based architecture for predicting pathogenicity of genomic mutations, in accordance with an embodiment. Given a test genomic mutation, the input of the network includes three types of annotations: DNA flanking sequence of the test mutation (primary annotation) as of step 410; and genetic or epigenetic or other features over the DNA flanking sequence of the test mutation (secondary annotation) as of step 420; and mutation-related or gene-level annotation (secondary annotation), represented as a scalar of numerical values as of step 430.

In some embodiments, the primary annotation is sequence data of a linear segment of genomic sequence from the human reference genome centered at the given genomic mutation. Initially, using the one-hot code, the sequence data can be encoded to a 4×L matrix, where L denotes the length of the sequence (e.g., 800 bp) and each column is a 4-element vector (e.g., [1, 0, 0, 0], [0, 1, 0, 0], [0, 0, 1, 0], [0, 0, 0, 1]) to indicate the presence or absence of A, C, G, or T at each nucleotide position.

In some embodiments, IUPAC code are used to encode ambiguous nucleotides. In detail, R (=G, A), Y (=T, C), M (=A, C), S(=G, C), W(=A, T), B(=G, T, C), D(=G, A, T), H(=A, C, T), V(G, C, A), N(A, C, G, T) are encoded as [1, 0, 1, 0], [0, 1, 0, 1], [1, 1, 0, 0], [0, 1, 1, 0], [1, 0, 0, 1], [0, 1, 1, 1], [1, 0, 1, 1], [1, 1, 0, 1], [1, 1, 1, 0], [1, 1, 1, 1], respectively.

In some embodiments, the secondary annotation, which is composed of annotation information across DNA flanking sequence of the test mutation, is represented as a data matrix as of step 420. In such a data matrix, each row denotes an annotated feature over the DNA flanking sequence, and each column denotes different annotated features at a nucleotide position or over a DNA segment. For example, a quantitative score reflects cross-species sequence conservation at a specific nucleotide, or a percentage number indicates the GC percentage of a 25 bp DNA segment.

In some embodiments, the secondary annotation is represented as a scalar of numerical values as of step 430, of which each numeric value indicates a specific annotated feature of the test genetic variation or a gene-level annotation.

The sub-CNN N1 shown in step 440 contains a number of convolutional filters with different window size in parallel, followed by a FC layer in the end. These convolutional filters are of different with size to learn from DNA flanking sequence at different spatial resolution. For instance, a convolution filter with window size 1 corresponds to learn from DNA sequence at single-nucleotide resolution; a convolutional filter with window size 2 indicates dinucleotide resolution; a convolutional filter with window size 3 indicates triple-nucleotide resolution or amino-acid resolution; a convolutional filter with window size 8 indicates 8mer resolution, where 8mer is the typical length of many DNA motifs. These convolution filters are further concatenated using a filter concatenation layer, followed by a FC layer to learn genetic correlative relationships among DNA flanking sequence of the test mutation. Step 450 illustrates an example of sub-CNN N2. Similar as sub-CNN N1 of step 410, sub-CNN N2 is composed of one or more convolutional filters (alternating convolutional layers and nonlinear pooling layers), followed by a fully connected (FC) layer in the end.

Sub-CNN N2 as in step 450 is composed of one or more convolutional filters, followed by a fully connected (FC) layer in the end.

Step 460 illustrates a merge layer to concatenate the resultant matrices or vectors from steps 440, 450, and 430 to a single data matrix or vector.

Sub-CNN N3 as in step 470 is similar as sub-CNN N2, which is composed of convolutional layers, followed by one or more FC layers.

In step 480, a non-linear transformation with sigmoid activation is used to transform the outputs of step 470 to a single value between 0 and 1, reflecting the pathogenic risk 490 of the give mutation.

FIG. 9B illustrates an exemplary implementation of sub-CNN N1 as of step 440. First, for each test mutation, the DNA flanking sequence of length 800 is extracted from human reference genome, and encoded using one-hot code as in step 410. The resultant input is a 800×4 matrix.

Second, four types of convolutional filter with different window sizes are used in parallel in step 400. The window sizes of these filters are 1×4, 2×4, 3×4, and 5×4, respectively. The number of filters for each convolution filter type is 64. Each type of convolution filters is followed by a max-pooling layer, which transforms the resultant 800×64 data matrix of each type of convolution filters to a vector with 800 numerical values. After these convolution layers, a filter concatenation layer merges the resultant 4 vectors to a single vector with length 3200. In the end, two FC layers are applied with sizes of 800 and 200, respectively. Therefore, the output of sub-CNN N1 module is a scalar of numerical values with length 200.

FIG. 9C illustrates an exemplary implementation of sub-CNN N2 and sub-CNN N3. In step 420, for the DNA flanking region of 800 bp around the test mutation, we retrieved (1) the layered H3K27Ac data (on 7 cell lines), (2) the layered H3K4Me1 data (on 7 cell lines), (3) the layered H3K3Me3 data (on 7 cell lines), (4) the DNase hypersensitivity cluster data (on 125 cell types), (5) the Transcription Factor ChIP-Seq (161 factors) from ENCODE project (Mar 2012 freeze), and calculated average scores for each non-overlapping nucleotide segment of 25 bp. Moreover, we retrieved (6) GC percent, (7) CpG island, and (8) 46-way mammal conservation data from UCSC genome browser (http://hgdownload.cse.ucsc.edu/goldenPath/hg19/bigZips/), and calculated average scores for each non-overlapping nucleotide segment of 25 bp. In summary, the secondary annotation in step 420 is a 32×8 numeric matrix, in which 32 denotes 32 non-overlapping 25 bp-long nucleotide segments in an 800 bp flanking region, and 8 denotes 8 annotated features across these segments.

The step 430 represents the secondary annotation data in the numerical scalar with 32 values. These values include (1) if mutation is in protein-coding region (binary); (2) mutation types (SNP/insertion/deletion/complex, a categorical variable with 4 factors); (3) Functional consequence of the test mutation (missense/in-frame/truncating/other, a categorical variable with 4 factors); (4) reparative sequence information at a 2000-bp region from RepeatMasker (SINE/LINE/LTR/DNA/Simple/Low Complex/Satellite/RNA/other/Unknown, a categorical variable with 10 factors), (5) FATHMM score (1 numerical value). In summary, the step 430 contains a numerical vector of length 32.

As shown in step 450, the sub-CNN N2 consists of three convolution layers, a convolution layer with eight 1×8 convolutional filters, followed by a convolution layer with eight 1×8 convolutional filters, and followed by a max-pooling layer. The output of this sub-CNN N2 is a numerical vector of length 32.

As shown in step 460, the merge layer concatenates three resultant vectors from the steps 430, 440, and 450 to a single vector of length 252.

As shown in step 470, the sub-CNN N3 module is composed of two FC layers, both with length 252. In step 480, a sigmoid activation layer is applied to transform the output of step 470 to a single numerical value 490, ranging from 0 to 1.

In some embodiments, the CNN model is trained using the standard back-propagation algorithm. In some embodiments, mini-batch gradient descent with AdaGrad optimization or an alternative technique is used to improve convergence performance, and dropout and early stopping is used to reduce overfitting.

Although the embodiments described in FIG. 9A, FIG. 9B and FIG. 9C are based on convolutional neural networks, the techniques disclosed herein can be implemented using other types of neural networks, for example, recursive neural networks (RNN) or multi-layer perceptrons. Furthermore, each sub-neural network can be a different type of neural network.

Computer Architecture

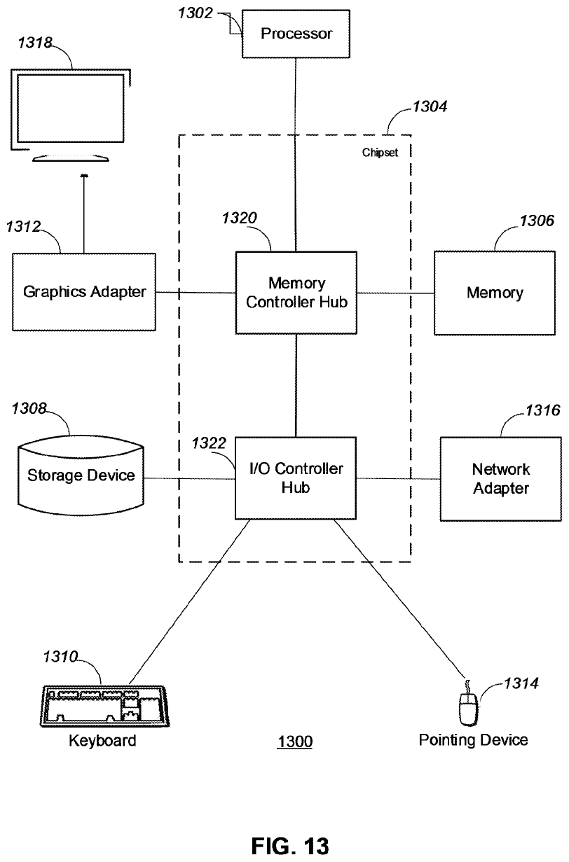

FIG. 13 is a high-level block diagram illustrating an example computer for implementing the client device and/or the computer system of FIG. 1. The computer 1300 includes at least one processor 1302 coupled to a chipset 1304. The chipset 1304 includes a memory controller hub 1320 and an input/output (I/O) controller hub 1322. A memory 1306 and a graphics adapter 1312 are coupled to the memory controller hub 1320, and a display 1318 is coupled to the graphics adapter 1312. A storage device 1308, an input device 1314, and network adapter 1316 are coupled to the I/O controller hub 1322. Other embodiments of the computer 1300 have different architectures.

The storage device 1308 is a non-transitory computer-readable storage medium such as a hard drive, compact disk read-only memory (CD-ROM), DVD, or a solid-state memory device. The memory 1306 holds instructions and data used by the processor 1302. The input interface 1314 is a touch-screen interface, a mouse, track ball, or other type of pointing device, a keyboard, or some combination thereof, and is used to input data into the computer 1300. In some embodiments, the computer 1300 may be configured to receive input (e.g., commands) from the input interface 1314 via gestures from the user. The graphics adapter 1312 displays images and other information on the display 1318. The network adapter 1316 couples the computer 1300 to one or more computer networks.

The computer 1300 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 1308, loaded into the memory 1306, and executed by the processor 1302.

The types of computers 1300 used by the entities of FIG. 1 can vary depending upon the embodiment and the processing power required by the entity. The computers 1300 can lack some of the components described above, such as graphics adapters 1312, and displays 1318. For example, the computer system 130 can be formed of multiple blade servers communicating through a network such as in a server farm.

Mutations

The methods for predicting pathogenicity described herein can be used for a broad range of genetic sequence variant types. In some embodiments the machine learning model is training using a genetic sequence variant data set comprising a broad range of genetic sequence variant types and is useful for predicting pathogenicity in a test genetic sequence variant with any genetic sequence variant. In some embodiments, the methods are more specialized for a particular genetic sequence variant type or a limited range of genetic sequence variant types. In such a specialized method, the machine learning model is trained using a genetic sequence variant training set comprising a limited number of genetic sequence variant types and is useful to predict the pathogenicity of a test genetic sequence variant comprising one of such genetic sequence variant types.

In some embodiments, the genetic sequence variant training data set comprises genetic sequence variants with a missense mutation, a nonsense mutation, a frame-shifting genetic sequence variant (such as an insertion genetic sequence variant or a deletion genetic sequence variant), a splice-site genetic sequence variant (such as a canonical splice-site genetic sequence variant or a non-canonical splice-site genetic sequence variant)), a coding region variant, an intronic region variant, a promoter region variant, an enhancer region variant, a 3′-untranslated region (3′-UTR) variant, a 5′-untranslated region (5′-UTR) variant, an intergenic region variant, a dominant genetic sequence variant, a recessive genetic sequence variant, or a loss-of-function (LoF) genetic sequence variant.

In some embodiments, the machine learning model is trained using a genetic sequence variant training data set comprising genetic sequence variants with a missense mutation. In some embodiments, the machine learning model is trained using a genetic sequence variant training data set comprising genetic sequence variants with a nonsense mutation. In some embodiments, the machine learning model is trained using a genetic sequence variant training data set comprising genetic sequence variants with a frame-shifting mutation. In some embodiments, the machine learning model is trained using a genetic sequence variant training data set comprising genetic sequence variants with a splice-site mutation.

In some embodiments, the machine learning model is trained using a genetic sequence variant training data set comprising genetic sequence variants with a mutation in a coding region. In some embodiments, the machine learning model is trained using a genetic sequence variant training data set comprising genetic sequence variants with a mutation in an intronic region. In some embodiments, the machine learning model is trained using a genetic sequence variant training data set comprising genetic sequence variants with a mutation in a promoter region. In some embodiments, the machine learning model is trained using a genetic sequence variant training data set comprising genetic sequence variants with a mutation in an enhancer region.

In some embodiments, the machine learning model is trained using a genetic sequence variant training data set comprising genetic sequence variants with a mutation in a 3′-untranslated region (3′-UTR). In some embodiments, the machine learning model is trained using a genetic sequence variant training data set comprising genetic sequence variants with a mutation in a 5′-untranslated region (5′-UTR).

In some embodiments, the machine learning model is trained using a genetic sequence variant training data set comprising genetic sequence variants with a mutation in an intergenic region. In some embodiments, the machine learning model is trained using a genetic sequence variant training data set comprising genetic sequence variants with a mutation in a regulatory region.

In some embodiments, the machine learning model is trained using a genetic sequence variant training data set comprising genetic sequence variants with a mutation in a dominant gene. In some embodiments, the machine learning model is trained using a genetic sequence variant training data set comprising genetic sequence variants with a mutation in a recessive gene. In some embodiments, the machine learning model is trained using a genetic sequence variant training data set comprising genetic sequence variants with a loss-of function mutation.

Pathogenicity

Although the systems and methods described herein are generally in the context of determining a pathogenicity of a mutant with respect to causation for cancer, the methods and systems described herein can be used to determine a pathogenicity of a mutant with respect to other diseases, disorders, and conditions as well. Thus, in some embodiments provided herein are methods of training and using machine learning systems to identify a pathogenicity associated with a mutation by using a primary annotation comprising a representation of the flanking sequence of the mutation and a secondary annotation comprising a representation of one or more other features associated with the mutation. In some embodiments, the machine learning system receives the primary annotation and the secondary annotation as discrete inputs, and converts them into a single output indicative of the pathogenicity of the mutation with respect to a disease, disorder, or condition of interest.

Methods of Use

The machine learning methods described herein provide a means to evaluate cancer-related pathogenic risk of any given genetic mutation.

In various embodiments, the methods are used to predict cancer-related pathogenic risk of de novo variants. The identification of novel cancer driver mutations and novel oncogenes unveils underlying biological processes and molecular mechanisms in carcinogenesis and cancer development. The newly identified cancer driver mutations and oncogenes may lead to identification of novel cancer drug targets.

With rapid advances in high-throughput sequencing technologies and development of targeted therapies, genomic testing has become the standard of care in directing treatment of cancer. The cancer-related mutations newly identified by the methods herein will aid in cancer panel design and evaluation of tumor mutational burden, thereby helping diagnosis and prognostic monitoring of cancer, and will help direct treatment options.

In particular, using cell-free DNA (cf-DNA) from blood to detect cancer and to monitor prognosis has become increasingly accepted. However, using cf-DNA to detect cancer at an early stage is still technically challenging, since the concentrations (intensities) of circulating tumor DNA (ct-DNA) are extremely low. Ultra-deep high-throughput sequencing approach has been proposed as a way to boost detection sensitivity. This approach faces two challenges. First, ultra-deep high-throughput sequencing means high cost. Second, the intensities of ct-DNA fragments among cf-DNA fragments are close to the error rates of the advanced high-throughput sequencing machines. Therefore, ultra-deep sequencing may not be able to distinguish real ct-DNA signals from the background sequencing errors.

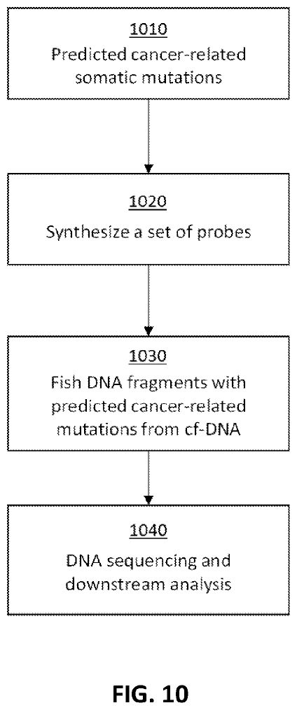

FIG. 10 illustrates an example process illustrating a method of using the predicted set of cancer-related somatic mutations to accurately and inexpensively detect tumor signals from blood, according to an embodiment. In the first step of the process, a set of nucleic acid probes is designed to detect the predicted high-risk cancer-related somatic mutations. The plurality of such probes are synthesized, each typically as single stranded DNA probe. Next, this probe set is used to selectively extract and isolate DNA fragments from cf-DNA, thereby enriching for DNA containing the cancer-related somatic mutations. Since the isolated cf-DNA fragments contain the predicted somatic mutations and the sequence intensities are relatively high, the cf-DNA can be sequenced with high confidence at a moderate depth.

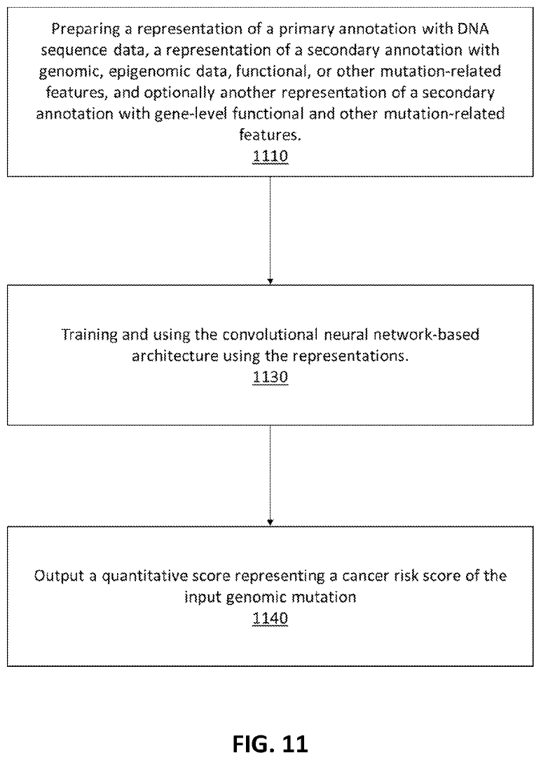

FIG. 11 illustrates an example process for predicting cancer-related pathogenic impact of somatic mutations using a convolutional neural network-based architecture, according to an embodiment. A first representation comprising a primary annotation with DNA sequence data, a second representation comprising secondary annotation of a mutation with genomic or epigenomic data, functional, or other mutation-related annotation, and optionally a third representation comprising a secondary annotation of a mutation with gene-level functional and other mutation-related features are prepared (1110). The representations are used as input for training and/or using a convolutional neural network-based architecture (1120). A score is generated from the architecture that is representative of a pathogenicity (e.g., a cancer risk score) corresponding to the input genomic mutation.

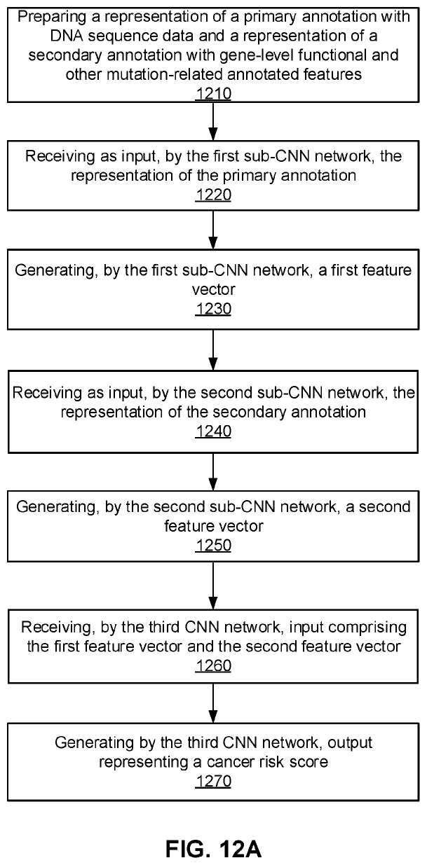

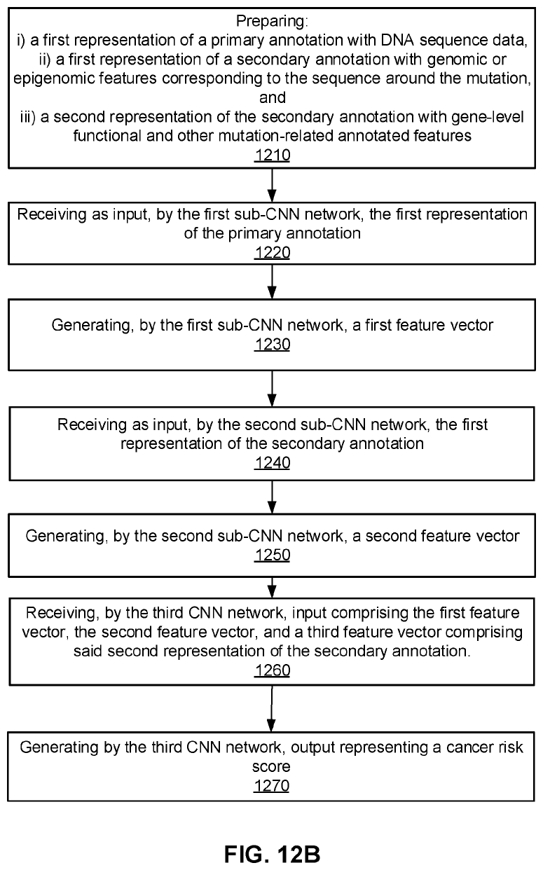

FIG. 12A illustrates an example process for predicting cancer-related pathogenic impact of somatic mutations using a convolutional neural network-based architecture, according to an embodiment. The input preparation module 1210 receives description of a genomic mutation as input and prepares vectors for providing as input to the CNN network based on the received description. The input preparation module 1210 processes the input description of the genomic mutation to prepare 1210 a representation of DNA sequence data (primary annotation) and a representation of the gene level functional features and other mutation-related features (secondary annotation). As shown in FIG. 4A, the CNN network includes a first sub-CNN network, a second sub-CNN network, and the third sub-CNN networks. The first sub-CNN network receives 1220 as input the representation of the DNA sequence data. The first sub-CNN network generates 1230 a first feature vector based on the input representation of the sequence data. The second sub-CNN network receives 1240 the representation of the secondary annotation as input. The second sub-CNN network generates 1250 a second feature vector based on the input representation of the secondary annotation. The third sub-CNN network receives 1260 as input the first feature vector and the second feature vector and generates 1270 an output representing the cancer risk score corresponding to the input sample.

FIG. 12B illustrates an example process for predicting cancer-related pathogenic impact of somatic mutations using a convolutional neural network-based architecture, according to an embodiment. The input preparation module 1210 receives description of a genomic mutation as input and prepares vectors or matrices for providing as input to the CNN network based on the received description. The input preparation module 1210 processes the input description of the genomic mutation to prepare 1210 a representation of DNA sequence data (primary annotation), a representation of a secondary annotation with genomic or epigenomic features corresponding to the sequence around the mutation, and a representation of a secondary annotation with the gene level functional features and other mutation-related features. As shown in FIG. 4B, the CNN network includes a first sub-CNN network, a second sub-CNN network, and the third sub-CNN networks. The first sub-CNN network receives 1220 as input the representation of the DNA sequence data. The first sub-CNN network generates 1230 a first feature vector based on the input representation of the sequence data. The second sub-CNN network receives 1240 the representation of the secondary annotation as input. The second sub-CNN network generates 1250 a second feature vector based on the input representation of the secondary annotation. The third sub-CNN network receives 1260 as input the first feature vector and the second feature vector and generates 1270 an output representing the cancer risk score corresponding to the input sample.

Those of skill in the art will recognize that other embodiments can perform the steps of FIGS. 12A-12B in different orders. Moreover, other embodiments can include different and/or additional steps than the ones described herein. Steps indicated as being performed by certain modules may be performed by other modules.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Example 1

### Deep-Learning Classification of Cancer Driver Mutations

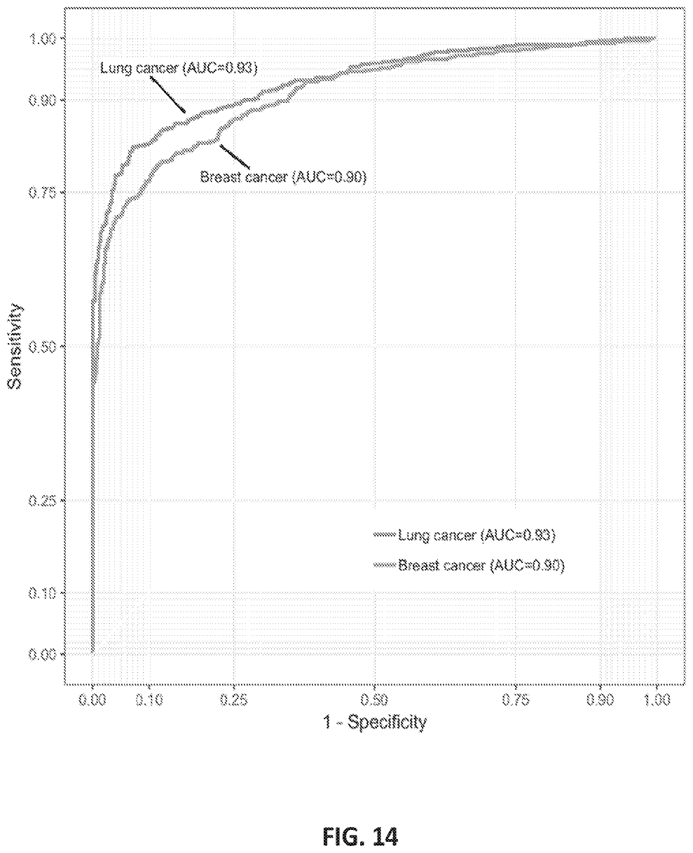

FIG. 14 demonstrates the performance of the deep-learning framework as described in FIGS. 9A, 9B and 9C, demonstrating its ability to distinguish cancer-causing somatic mutations from common mutations in two cancer types, lung cancer and breast cancer.

The positive dataset, which contained confirmed or validated cancer-associated somatic mutation, was retrieved from data repositories of COSMIC (v85), ClinVar (dated Dec. 31 , 2017), and Emory Genetics Laboratory (EmVClass, dated 2018 Q1). In detail, the mutations labelled as “confirmed” in COSMIC and “pathogenic or likely pathogenic in cancer” in ClinVar and EmVClass were categorized as high-confidence cancer driver mutations and were included in the positive dataset. The positive dataset was further filtered by cancer type (e.g., breast cancer and lung cancer). Eventually, we obtained two positive datasets, one for breast cancer with approximately 20,000 single-nucleotide variant (SNVs) and small indels (less than 10 bp), and the other one for lung cancer with approximately 27,000 SNVs and small indels.

The control dataset, which contained confirmed or validated common variants or confirmed non-cancer-associated or benign mutations, was retrieved from the dbSNP database (build 150). The mutations with a minor allele frequency less than 1% were excluded. The mutations that were included in the positive dataset were excluded. The mutations that are more than 2,000 bp away from any mutations in the positive dataset were excluded. In sum, the negative dataset contained approximately 200,000 mutations.

To keep positive and control datasets balanced, we over-sampled 50,000 mutations from the positive dataset and down-sampled 50,000 mutations from the control dataset. Furthermore, the positive and control datasets were split into two parts with 80% and 20% of mutations, respectively. The datasets with 80% of mutations were used in the training procedure (training datasets), and the remaining datasets with 20% of mutation data were used as test datasets.

In order to improve convergence performance and to further reduce the variance of the gradient, we applied mini-batch gradient descent with AdaGrad optimization. To avoid overfitting, we applied dropout (rate 0.5) and early stopping at each of the convolution layers.

Receiver Operating Characteristic (ROC) curves, as shown in FIG. 14, were used to evaluate prediction performance of the CNN-based models. The values of the area under the ROC curve (AUC) for breast and lung cancers were 0.90 and 0.93, respectively, suggesting superior predictive power of the proposed deep-learning framework.

### Alternate Embodiments

It is to be understood that the Figures and descriptions of the present invention have been simplified to illustrate elements that are relevant for a clear understanding of the present invention, while eliminating, for the purpose of clarity, many other elements found in a typical distributed system. Those of ordinary skill in the art may recognize that other elements and/or steps are desirable and/or required in implementing the embodiments. However, because such elements and steps are well known in the art, and because they do not facilitate a better understanding of the embodiments, a discussion of such elements and steps is not provided herein. The disclosure herein is directed to all such variations and modifications to such elements and methods known to those skilled in the art.

Some portions of above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for displaying charts using a distortion region through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A computer-implemented method for predicting a pathogenicity of a test genomic mutation, comprising:
preparing input data for said test genomic mutation, comprising
generating a first representation comprising a primary annotation of said test genomic mutation, wherein said primary annotation comprises a nucleotide sequence surrounding said test genomic mutation;
generating a second representation comprising at least one feature of a secondary annotation of said test genomic mutation, wherein said feature comprises a functional feature, a genomic feature, or an epigenomic feature;
generating a score from said first and second representations of said test genomic mutation using a convolutional neural network (CNN), wherein said convolutional neural network comprises a first sub-CNN network configured to process the first representation, a second sub-CNN network configured to process the second representation, and a third sub-CNN network configured to process the output of the first and second sub-CNN network, wherein generating said score comprises
receiving, by the first sub-CNN network, said first representation of said test genomic mutation;
generating, by the first sub-CNN network, output comprising a first feature vector;
receiving, by the second sub-CNN network, said second representation of said test genomic mutation;
generating, by the second sub-CNN network, output comprising a second feature vector;
receiving, by the third sub-CNN network, input comprising the first feature vector and the second feature vector; and
generating, by the third sub-CNN network, output representing a score indicative of a pathogenicity associated with the test genomic mutation.

2. The method of claim 1, wherein the third sub-CNN network comprises a fully connected layer.

3. The method of claim 1, wherein said pathogenicity is a cancer risk from said mutation.

4. The method of claim 1, further comprising generating a third representation comprising at least one feature of said secondary annotation of said test genomic mutation.

5. The method of claim 4, further comprising receiving, by the third sub-CNN network, input comprising the third representation.

6. The method of claim 4 or 5, wherein said third representation is a vector.

7. The method of claim 6, wherein said vector comprises functional features corresponding to said mutation.

8. The method of any one of claims 1-5, wherein said second representation is a matrix.

9. The method of claim 8, wherein said matrix comprises genomic or epigenomic feature information corresponding to said surrounding nucleotide sequence.

10. The method of any one of claims 1-5, wherein said first representation is a vector.

11. The method of claim 10, wherein said vector comprises said surrounding nucleotide sequence.

12. The method of any one of claims 1-5, wherein said convolutional neural network is trained based on training data comprising:
a first data set comprising pathogenic mutations, and
a second data set comprising benign mutations.

13. The method of claim 12, wherein said pathogenic mutations are cancer driver mutations.

14. The method of claim 12, wherein said training data comprises said first representation and said second representation for each mutation in said first data set and said second data set.

15. The method of claim 14, wherein said training data further comprises said third representation for each mutation in said first data set and said second data set.

16. The method of claim 1, further comprising storing said test genomic mutation in a database if said score indicates a high pathogenicity risk associated with the mutation.

17. The method of any one of claims 1-5, further comprising training a convolutional neural network based on a training data set, said training data comprising:
a first data set comprising said first representation and said second representation for each of a plurality of pathogenic mutations, and
a second data set comprising said first representation and said second representation for each of a plurality of benign mutations.

18. The method of claim 17, wherein said first data set and said second data set each further comprise said third representation.

19. A computer-implemented method for predicting a pathogenicity of a test genomic mutation, comprising:
preparing input data for said test genomic mutation, comprising:
generating a first representation comprising a primary annotation of said test genomic mutation, wherein said primary annotation comprises a nucleotide sequence surrounding said test genomic mutation;
generating a second representation and a third representation each comprising one or more features of a secondary annotation of said test genomic mutation, wherein said secondary annotation comprises functional, genomic, or epigenomic features, wherein said second representation comprises features corresponding to said nucleotide sequence surrounding said test genomic mutation, wherein said third representation comprises functional features corresponding to said test genomic mutation;
generating a score from said first, second, and third representations of said test genomic mutation using a convolutional neural network (CNN), wherein said convolutional neural network comprises a first sub-CNN network configured to process the first representation, a second sub-CNN network configured to process the second representation, and a third sub-CNN network configured to process the output of the first and second sub-CNN network and the third representation, wherein generating said score comprises:
receiving, by the first sub-CNN network, said first representation of said test genomic mutation;
generating, by the first sub-CNN network, output comprising a first feature vector;
receiving, by the second sub-CNN network, said second representation of said test genomic mutation;
generating, by the second sub-CNN network, output comprising a second feature vector;
receiving, by the third sub-CNN network, input comprising the first feature vector, the second feature vector, and said third representation of said test genomic mutation; and
generating, by the third sub-CNN network, output representing a score indicative of a pathogenicity associated with the test genomic mutation.

20. A computer-implemented method of predicting a pathogenicity score of a test genomic mutation, comprising:
preparing input data for said test genomic mutation, comprising
generating a first representation comprising a primary annotation of said test genomic mutation, wherein said primary annotation comprises a nucleotide sequence surrounding said test genomic mutation; and
generating a second representation comprising a feature of a secondary annotation of said test genomic mutation, wherein said secondary annotation comprises functional, genomic, or epigenomic features;
providing the first and second representation as input to a trained machine
learning model, wherein the machine learning model is configured to
receive input comprising said first representation and said second representation; and
generate a score indicative of a pathogenicity risk associated with the test genomic mutation.

21. The method of claim 20, wherein said score is a cancer risk score.

22. The method of claim 20, wherein preparing said input data further comprises generating a third representation comprising a functional feature of said secondary annotation.

23. The method of claim 22, further comprising providing the third representation as input to said trained machine learning model, said machine learning model configured to receive said third representation as input.

24. The method of any one of claims 20-23, wherein said first representation is a vector comprising said surrounding nucleotide sequence.

25. The method of any one of claims 20-23, wherein said second representation is a vector.

26. The method of any one of claims 20-23, wherein said second representation is a matrix.

27. The method of claim 26, wherein said matrix comprises epigenomic or genomic feature information corresponding to said surrounding nucleotide sequence.

28. The method of any one of claim 22 or 23, wherein said third representation is a vector.

29. The method of claim 28, wherein said vector comprises functional features corresponding to said mutation.

30. The method of any one of claims 20-23, wherein the machine learning model is trained based on training data comprising
a first data set comprising pathogenic mutations, and
a second data set comprising benign mutations.

31. The method of claim 30, wherein said pathogenic mutations are cancer driver mutations.

32. The method of claim 30, wherein said training data comprises said first representation and said second representation for each mutation in said first data set and said second data set.

33. The method of claim 32, wherein said training data set comprises said third representation for each mutation in said first data set and said second data set.

34. The method of claim 20, further comprising storing said test genomic mutation in a database if said score indicates a high cancer risk associated with the mutation.

35. The method of any one of claims 20-23, further comprising training a machine learning model based on a training data set, said training data comprising
a first data set comprising said first representation and said second representation for each of a plurality of pathogenic mutations, and
a second data set comprising said first representation and said second representation for each of a plurality of benign mutations.

36. The method of claim 35, wherein said pathogenic mutations are cancer driver mutations.

37. The method of claim 35, wherein said first and second data set further comprise said third representation for each of said mutations.

38. A computer-implemented method of predicting a cancer risk score of a test genomic mutation, comprising:
preparing input data for said test genomic mutation, comprising
generating a first representation comprising a primary annotation of said test genomic mutation, wherein said primary annotation comprises a nucleotide sequence surrounding said test genomic mutation;
generating a second representation comprising one or more features of a secondary annotation of said test genomic mutation, wherein said secondary annotation comprises functional, genomic, or epigenomic features corresponding to said nucleotide sequence surrounding said test genomic mutation;
generating a third representation comprising one or more functional features of said secondary annotation of said test genomic mutation;
providing the first, second, and third representations as input to a trained machine learning model, wherein the machine learning model is configured to
receive input comprising said first representation and said second representation, and
generate a score indicative of a cancer risk associated with the test genomic mutation.

39. The method of any of the above claims, further comprising using said score to identify said cancer risk associated with the test genomic mutation.

40. The method of any of the above claims, wherein said score is used to identify a cancer driver mutation.

41. The method of any one of the above claims, further comprising repeating said method to identify a plurality of cancer driver mutations.

42. The method of any of the above claims, further comprising sending said score for display via a user interface.

43. The method of any of the above claims, further comprising transforming the score to a defined range using a sigmoid function.

44. The method of claim 43, wherein said defined range is from 0 to 1.

45. The method of any of the above claims, wherein said pathogenicity is a cancer risk associated with a specific tissue type.

46. The method of any of the above claims, wherein said pathogenicity is a cancer risk associated with a specific cancer type.

47. The method of any of the above claims, further comprising performing said method on a plurality of test genomic mutations.

48. The method of any of the above claims, wherein the test genomic mutation is a human genomic mutation.

49. The method of any of the above claims, wherein the test genomic mutation is a somatic mutation.

50. The method of any of the above claims, wherein the test genomic mutation comprises a missense genomic mutation, a nonsense genomic mutation, a splice-site genomic mutation, an insertion genomic mutation, a deletion genomic mutation, or a regulatory element genomic mutation.

51. The method of any of the above claims, wherein the test genomic mutation comprises a single nucleotide polymorphism.

52. The method of any of the above claims, wherein the secondary annotation comprises a protein level functional impact feature, a region identifier feature, a sequence conservation feature, an epigenetic status feature, a genomic feature of the surrounding sequence, or any combination thereof.

53. The method of any of the above claims, wherein the pathogenicity is a cancer risk that is predictive of the probability that a mutation is causative for cancer.

54. A non-transitory computer-readable storage medium comprising computer-executable instructions for carrying out any one of the above claims.

55. A system comprising
one or more processors;
memory; and
one or more programs, wherein the one or more programs are stored in the memory and configured to be executed by the one or more processors, the one or more programs including instructions for carrying out any one of the above claims.

56. A system comprising a convolutional neural network (CNN), said CNN comprising
a first sub-CNN network configured to
receive as input a first representation comprising a primary annotation of a test genomic mutation, wherein said primary annotation comprises a nucleotide sequence surrounding said test genomic mutation, and
generate as output a first feature vector;
a second sub-CNN network configured to
receive as input a second representation comprising at least one feature of a secondary annotation of said test genomic mutation, wherein said feature comprises a functional feature, a genomic feature, or an epigenomic feature, and
generate as output a second feature vector; and
a third sub-CNN network configured to merge the output of the first and
second sub-CNN network by
receiving, as input
a first feature vector generated by the first sub-CNN network, and
a second feature vector generated by the second sub-CNN network; and
generating as output a score indicative of a cancer risk associated with the test genomic mutation.

57. The system of claim 56, wherein third sub-CNN network is configured to merge the output of the first and second sub-CNN network and a third representation comprising at least one feature of said secondary annotation.

58. A set of probes configured to bind specifically to a plurality of oligonucleotides, each oligonucleotide comprising at least one of said plurality of cancer mutations identified by the method of any one of claims 1-53.

59. The set of probes of claim 58, wherein said plurality of oligonucleotide are cf-DNA fragments.

60. A method of diagnosing, monitoring, or determining the state of cancer in a subject, comprising:
contacting the set of probes of claim 58 or 59 to a sample from a subject diagnosed with or suspected of having cancer; and
detecting the identity of mutations bound to the probes to diagnose, monitor, or determine the state of cancer in said subject.
